(19) United States
(12) Patent Application Publication
(10) Pub. No.: US 2024/0077481 A1
(43) Pub. Date: Mar. 7, 2024

(54) IMPROVED TREATMENT METHODS USING DMDS FOR THE TREATMENT OF AUTOIMMUNE DISEASES, AND BIOMARKER FOR PREDICTING AND/OR OPTIMISING SAID TREATMENT METHODS

(71) Applicant: ARES TRADING S.A., AUBONNE (CH)

(72) Inventor: URSULA BOSCHERT SHAFAATIAN, EYSINS (CH)

(21) Appl. No.: 18/280,340

(22) PCT Filed: Mar. 3, 2022
(86) PCT No.: PCT/EP2022/055484

§ 371 Date: Sep. 5, 2023

Related U.S. Application Data
(60) Provisional application No. 63/156,196, filed on Mar. 3, 2021.
(60) Provisional application No. 63/174,949, filed on Apr. 14, 2021.

(51) Int. Cl. G01N 33/569; G01N 33/564

(52) U.S. Cl. CPC G01N 33/56972; G01N 33/564; G01N 2800/285; G01N 2800/52; G01N 2800/54

(57) ABSTRACT

The invention pertains to improved treatment methods using DMDS, in particular cladribine, for the treatment of autoimmune diseases, in particular multiple sclerosis, and biomarkers, in particular immune cell subtypes, for predicting and/or optimising said treatment methods. The methods described rely on the use of immune cell subtypes, in particular T cells, B cells, NK cells subtypes and their ratios as present in blood samples from the patients for predicting and/or optimising the use of cladribine in the treatment of multiple sclerosis.

### INTRODUCTION/BACKGROUND OF THE INVENTION/BRIEF SUMMARY OF THE INVENTION

Multiple sclerosis (MS) is characterized by the presence of focal and diffuse inflammatory and degenerative lesions in the CNS. Continuous modulation of the immune system, e.g. by DMDs (Disease Modifying Drugs), is potentially able to modify disease activity and can also potentially influence the disease course, especially in early phases. The therapeutic armamentarium has developed substantially over the past two decades, and now includes ˜16 different drugs, preferably Disease Modifying Drugs. In 2017 the European Medicines Agency (EMA) has recommended the approval of cladribine or, more specific, oral cladribine or cladribine tablets, as an orally administered immunosuppressive drug, for the treatment of highly active MS. Development of cladribine, preferably oral cladribine or cladribine tablets, began after the observation that inherited deficiency of adenosine deaminase (ADA) an enzyme involved in purine metabolism causes lymphopenia in humans and leaves other tissues unaffected, owing to intracellular accumulation of toxic deoxyadenosine nucleotides specifically within lymphocytes. Lymphocytes exhibit high levels of deoxycytidine kinase activity and low levels of nucleotidase activity, which means that they rely on ADA activity to remove excess triphosphorylated nucleotides. Consequently, ADA-resistant purine nucleoside analogues such as cladribine, can replicate the lymphopenic effects of ADA deficiency. Upon entering lymphocytes, ADA-resistant cladribine is triphosphorylated by deoxycytidine kinase to generate its lymphocytotoxic form, resulting in selective diminution of the lymphocyte population.

Some DMDs are to be administered as chronic therapies wherein the drug is continuously administered in order to reach optimized reduction of disease activity. Some other DMDs, for example Cladribine, preferably oral cladribine or cladribine tablets, is given in short treatment courses of 2 weeks in year 1 and 2, preferably as described in the posology section of the EU EMA SmPC and/or the US/FDA USPI for oral cladribine or cladribine tablets, or an equivalent manner thereof, allowing immune reconstitution that is capable of (transiently or permanently) induce an immune reset in patients treated accordingly with cladribine, preferably oral cladribine or cladribine tablets, with the potential to produce long-lasting drug-free remission.

This important information was obtained from the CLARITY extension study, which investigated the continued efficacy of cladribine, preferably oral cladribine or cladribine tablets, after the initial CLARITY trial. In this study, placebo-treated patients from the CLARITY trial received a cumulative dose of 3.5 mg/kg cladribine, preferably oral cladribine or cladribine tablets, in the extension. Patients who had originally received cumulative doses of 3.5 mg/kg or 5.25 mg/kg cladribine, preferably oral cladribine or cladribine tablets, were randomly reassigned in a ratio of 2:1 to receive 3.5 mg/kg cladribine, preferably oral cladribine or cladribine tablets, or a placebo, producing five patient groups in total. Interestingly, relapse rates and disability were not significantly different between all groups, indicating that the efficacy of cladribine, preferably oral cladribine or cladribine tablets, is maintained in the long-term, irrespective of the order in which treatment, preferably treatment with cladribine, oral cladribine or cladribine tablets, is received. These results demonstrated that a short ‘pulse-like’ course of cladribine, preferably oral cladribine or cladribine tablets, delivered in two cycles could generate long-lasting lymphopaenia and/or response/efficacy, and potentially maintain very durable drug-free remission.

In the treatment of MS and preferably also other inflammatory and/or demyelinating autoimmune disorders, cladribine, preferably oral cladribine or cladribine tablets, is advantageously found to be capable to provide highly efficacious immune reconstitution therapies of such disorders when used for short, intermittent periods, thereby preferably inducing immune resetting and allow for treatment-free periods, preferably prolonged treatment-free periods, potentially prolonged treatment-free periods lasting for 2 to 5 years or 3 to 4 years, and potentially beyond, counted from the last administration of cladribine, preferably oral cladribine or cladribine tablets, to said patients. Long-term lymphocyte dynamics have been evaluated for CLARITY, CLARITY Extension, and PREMIERE studies, potentially indicating some kind of immune cell repopulation after treatment with cladribine tablets.

The design of the MAGNIFY-MS Study aims to determine the onset of action of cladribine tablets (3.5 mg/kg cumulative dose over 2 years) in patients with highly active relapsing MS. Surprisingly, it was now found that the action of cladribine, preferably oral cladribine or cladribine tablets, on immune cells may be key for both onset and durability of its effect in people with MS and preferably also other inflammatory and/or demyelinating autoimmune disorders. Whether and how cladribine, preferably oral cladribine or cladribine tablets, rebalances, reshapes or even permanently reprograms the immune system has previously not been or at least incompletely been understood. We are preferably addressing this lack of understanding in this invention.

The use of disease-modifying therapies in autoimmune conditions is of great clinical interest; however these therapies suffer from the inability to determine in a reasonable manner, e.g. using a test or preferably a blood test, whether a patient's immune system is responding appropriately to treatment or not. In the current invention we preferably propose the use of various Immune subtype based efficacy or retreatment biomarkers, preferably prognostic and/or predictive biomarkers, either alone or in combination. Preferably, these biomarkers are readily available in body fluid samples of said individuals and/or patients, preferably individuals or patients to be potentially treated or re-treated with one or more DMDs, preferably including but not limited to cladribine, preferably oral cladribine and/or cladribine tablets.

### SUMMARY OF THE INVENTION/DETAILED DESCRIPTION OF THE INVENTION

The meanwhile globally health authority approved use of cladribine tablets, as outlined in the EU EMA SmPC and/or US/FDA USPI, follows a unique posology according to which about 0.875 mg per kilogram of the bodyweight of the respective patient is administered within the first week of the first month (first treatment month) in which cladribine tablets treatment is started, followed by an additional administration of about 0.875 mg per kilogram of the bodyweight of the respective patient within the first week of the second month (second treatment month) in which cladribine tablets treatment is started, followed by about 10 months wherein no cladribine tablets or other DMD is administered to said patient (which period is preferably referred to as the first treatment year), followed by a further administration of about 0.875 mg per kilogram of the bodyweight of said patient within the first week of the month that follows the about 10 months in which no cladribine tablets or other DMD has been administered to said patient (e.g. thirteenth treatment month or first treatment month in the second treatment year) in which cladribine tablets treatment is started, finally followed by another administration of about 0.875 mg per kilogram of body weight within the first week of the month following the 13^{th} treatment month/1^{st} treatment month in the second treatment year. This unique treatment regimen has proven high efficacy in the treatment of MS, preferably relapsing-remitting MS (RRMS), i.e. by both a dramatic reduction of relapse rates and/or MRI activity in the CNS of the patients treated. Moreover, the high efficacy of this treatment regimen/posology for cladribine tablets as outlined above is maintained within the 10 months after the last treatment in said second treatment year and has been documented to be sustained in the year following the second treatment year (i.e. year three counted from the start of cladribine tablets treatment) and also in the subsequent year (i.e. year far counted from the start of cladribine tablets treatment), and beyond. Accordingly, a plurality of patients having received cladribine tablets treatment in year one (first treatment year) and here to (second treatment year) have such reduced MS disease activity in years 3 to 6 (and potentially beyond) following the start of initial cladribine tablets treatment that they do not require retreatment with cladribine tablets are any other DMD.

However, even though both the treatment regimen/posology and/or the high efficacy associated therewith are comparable to, if not better than, those associated with other DMD's, a detailed analysis of the efficacy in the treated patient collective shows that there are patients that in fact benefit more or less on the treatment with cladribine tablets as described above, both with regard to the onset or early efficacy of action and duration of efficacy or long-term efficacy. On one hand, this implies that there may be patients that potentially would benefit from an intensified treatment with cladribine tablets, e.g. an early or earlier retreatment with cladribine tablets (earlier than according to the treatment regimen/posology as described above) or an early switch to a different DMD, such as a cladribine tablets retreatment or switch to a different within the first and/or second treatment year of cladribine tablets treatment as described above. On the other hand, this further implies that there may be patients that are potentially prone to MS disease reactivation towards the end of the second treatment year with regards to cladribine tablets treatment, or e.g. in the subsequent 1-6 years following the second treatment year with regard to cladribine tablets treatment, which again could strongly benefit from a comparatively early retreatment with cladribine tablets or a comparatively early switch to a different DMD. Likewise, physicians and/or patients might consider a comparatively early retreatment with cladribine tablets or a comparatively early switch to another DMD in order to prevent potential disease reactivation, thereby risking potential harm to the patient retreated or retreated early by the respective DMD that is administered to said patient without an actual need for such a retreatment. For example, the patient data shows that there are patients that appear not to need cladribine tablets and/or other DMD treatment in the future, at least the foreseeable future, once treated with cladribine tablets in years one and two according to the posology/dosing regimen given above.

Thus, it would be highly advantageous if there would be a method to predict the patients response to treatment with cladribine tablets, both with regards to efficacy in general, including early to mid-term efficacy, and long-term efficacy, especially towards the end of the second treatment year, and the years following the end of said second treatment year.

This high medical need is preferably successfully addressed by the instant invention. According to the instant invention, certain deviations with regard to one or more cell populations, preferably one or more lymphocyte cell populations, preferably cell populations as described in more detail herein, are determined in one or more body fluids of diseased person or patient at different stages of treatment, preferably including determining said cell population at least once before the start of treatment with disease-modifying drug (DMD), and at least once at a later point in time during or after treatment with said DMD, wherein the differences or deviations of said cell populations determined at the different time points are indicative of efficacy of treatment, preferably including early onset of efficacy, mid-term efficacy, long-term efficacy and/or sustained efficacy. However, said differences or deviations of said cell populations determined at the different time points are preferably also indicative of non-efficacy of treatment, preferably including lack of onset of (early) efficacy, loss of (early) efficacy, lack of mid-term efficacy, loss of mid-term efficacy, lack of long-term efficacy and/or loss of long-term efficacy.

Thus, said methods preferably include determining said cell populations before start of treatment with said DMD, and at least at one further point in time, selected from, e.g., during treatment with said DMD, after treatment with said DMD, before re-treatment with said DMD, during re-treatment, and after re-treatment with said DMD. Typically, determining a said cell population (again) at at least one further point in time, preferably at an early stage of treatment of said treatment of said autoimmune disorder with a disease-modifying drug (DMD), preferably provides for a prediction or evidence of early efficacy and/or early onset of efficacy of treatment of said autoimmune disorder with said DMD, or a lack of early efficacy and/or early onset of efficacy under these conditions. Typically, the method according to the invention preferably provides for a method to predict the patient's response, preferably early response, to said treatment with a DMD, when said one or more cell populations are determined before the start of the treatment with said DMD (i.e. at baseline or baseline level), and determined again during or after said treatment with said DMD, preferably within 1 to 12 months from the start of said treatment with said DMD, more preferably 1 to 9 months from the start of said treatment with said DMD or even more preferably 1 to 6 months from the start of said treatment of said DMD, e.g. determining said one or more cell populations (again) in months 1, month 3 or month 6. As outlined in more detail below, the deviation between said one or more cell populations at the different time points, or the deviation between the determinations thereof at different timepoints, are indicative of efficacy or lack of efficacy, preferably indicative of early efficacy or lack of early efficacy. In case of indication of efficacy, the physician will appreciate that there is no need for a retreatment of said patient with the same DMD, the administration of an additional and/or higher dose of the same DMD, or no need to switch to a different DMD. In case, however, that there is an indication of lack of efficacy, the physician will appreciate the (early) information, that an additional and/or higher dose of the same DMD, a switch to a different DMD, and/or the addition of a further DMD will be beneficial for said patient.

Likewise, the method according to the invention preferably provides for a method to predict the patient's response, preferably long-term response, to said treatment with a DMD, when said one or more cell populations are determined before the start of the treatment with said DMD (i.e. at baseline or baseline level), and determined again during or after said treatment with said DMD, preferably after said treatment with said DMD, preferably within 12 to 60 months or 14 to 16 months from the start of the first treatment with said DMD, or preferably within preferably within 1 to 48 months from the last treatments with said DMD, or more specifically, from the day of the last administration of said DMD to said patient. The further determination of said one or more cell population can be repeated in regular intervals, e.g. intervals of 6 to 24 months, more preferably 9 to 18 months, e.g. about once a year. By said determination of said one or more cell populations (again) as outlined above and/or below, the mid-term and/or long-term efficacy of said treatment As outlined in more detail below, the deviation between said one or more cell populations, or the deviation between the determinations thereof, are indicative of efficacy or lack of efficacy, preferably indicative of early efficacy or lack of early efficacy. In case of indication of efficacy, preferably mid-term efficacy and/or long-term efficacy, the physician will appreciate that there is no need for a retreatment of said patient with the same DMD, the administration of an additional and/or higher dose of the same DMD, or no need to switch to a different DMD. In case, however, that there is an indication of lack of efficacy, the physician will appreciate the (early) information, that an additional and/or higher dose of the same DMD, a switch to a different DMD, and/or the addition of a further DMD, and especially a re-treatment with the same DMD will be beneficial for said patient. For example, in the treatment of MS with the DMD cladribine or cladribine tablets according to the health authority approved treatment scheme (EMA SmPC in force from 2017 to date (2021); FDA USPI in force from 2019 to date (2021)) with two short treatment periods of about a month each from the beginning of the treatment with cladribine (first treatment year), with two further short treatment periods of about a month each (second treatment year) starting at about the first anniversary of the beginning of the treatment with cladribine or cladribine tablets, most patients do not require retreatment with cladribine in tablets before the end of the fourth year from the beginning of the fourth or fifth anniversary of the beginning of the treatment with cladribine tablets, and often beyond. However, some patients might benefit from an earlier re-treatment with cladribine or cladribine tablets. Thus, the method according to the invention would provide early information to the physician that retreatment would be beneficial at a point in time, where no disease activity or relapses are yet detectable. This could be achieved by performing the method according to the invention one or more times, such as within the first twelve month from the start of said DMD treatment, or within months 12 to 60, months 24 to 60 and especially months 36 to 60, from the start of said DMD treatment.

Thus, preferred aspects of the instant invention include, but are not limited to:
A. Biomarkers (Immune signatures) for disease management of autoimmune diseases, preferably autoimmune neurological and neuroinflammatory diseases, preferably including monitoring early (preferably months 1-3) and long-term response (preferably months 6-12-24 and beyond, e.g. 3 to 15 years, preferably up to 3, 4, 5, 6, 7, 8, 9 or 10 years, of said autoimmune diseases, preferably autoimmune neurological diseases, even more preferably in the context of cladribine treatment.

As will be further defined herein, the immune signatures of the invention defined by the presence and/or relative quantity of certain subpopulations of immune cells in the patient, more particularly as identified and/or quantified from a biological sample of the patient.

By “biological sample”, it is herein referred to a sample obtained from a human subject. A biological sample may comprise tissues and/or biological fluids. As a non-limiting example, the biological sample may be selected from tissues, organs, cells, or any isolated fraction of a human subject, such as for instance from blood, plasma, lymph, saliva, urine, stool, tears, sweat, sperm, or cerebrospinal fluid, synovial, pleural, peritoneal, or pericardial, and any fraction or extracts thereof. Preferably, the biological sample is a biological fluid that comprises immune cells, preferably circulating immune cells. More preferably, the biological sample is blood, plasma or serum. The biological sample may be pre-processed to preserve the integrity of the cells of interests and/or to make them more accessible for further analysis. The biological sample may for instance undergo centrifugation, purification, or other treatment steps to facilitate access or increase the concentration of cells of interest. A blood sample may for instance be pre-processed through the use of a density gradient centrifugation process or by leukapheresis, using conventional techniques, to concentrate or isolate Peripheral Blood Mononuclear Cells (PBMCs).

Methods to identify and number immune cells or populations of immune cells are well known in the art as phenotyping or immunophenotyping, and are typically based on flux cytometry, also herein designed as FACs or FACs analysis. Immunophenotyping is the analysis of heterogeneous populations of cells for the purpose of identifying the presence, absolute number and/or and proportions of the various populations of interest. Fluorophore-conjugated antibodies are used to identify, characterize and quantify distinct subpopulations of cells within heterogeneous single-cell populations by detecting specific antigens, also herein designed as biomarkers, expressed by these cells (usually functional membrane proteins involved in cell communication, adhesion, or metabolism). Briefly, a sample expected to comprise immune cells, such as a biological sample, is put in contact with fluorophore-conjugated antibodies specific of the biomarkers expected to be either present on the cell surface or secreted by the cell. The sample is then analyzed using an optical, laser-based instrument which analyzes the physical and fluorescent properties of cells in suspension in real-time as they flow through the instrument. Such methods are conventional in the art and have been described in detail, using different fluorophores, in different publication to which the person skilled in the art can refer, such as articles published by Pitoiset et al. (Deep phenotyping of immune cell populations by optimized and standardized flow cytometry analyses. Cytometry A. 2018 August; 93(8):793-802. doi: 10.1002/cyto.a.23570. Epub 2018 Aug. 31. PMID: 3016889), by Streitz et al. (Standardization of whole blood immune phenotype monitoring for clinical trials: panels and methods from the ONE study. Transplant Res. 2013 Oct. 25; 2(1):17. doi: 10.1186/2047-1440-2-17. PMID: 24160259; PMCID: PMC3827923) or Hasan et al. (Milieu Interieur Consortium. Semi-automated and standardized cytometric procedures for multi-panel and multi-parametric whole blood immunophenotyping. Clin Immunol. 2015 April; 157(2):261-76. doi: 10.1016/j.clim.2014.12.008. Epub 2015 Jan. 6. PMID: 25572534).

In the context of the invention, the terms “peripheral blood mononuclear cells”, also “PBMSc” or “total PBMCs”, are to be construed as generally understood in the field, that is to say any and all peripheral blood cell having a round nucleus, i.e. lymphocytes (T cells, B cells, NK cells) and monocytes. Methods to isolate PBMCs are well known in the art, and these cells can be obtained from whole blood, diluted with PBS, gently layered over an equal volume of Ficoll in a Falcon tube and centrifuged for 30-40 minutes at 400-500 g without brake. Four layers will form, each containing different cell types the uppermost layer will contain plasma, which can be removed by pipetting. The viability, concentration and/or absolute numbers of isolated PBMCs can be measured by manual counting with trypan blue (TB) using a hemacytometer, or alternatively using an automated cell counter.

In the context of the invention, the term “B cells” is to be construed as generally understood in the art, that is to say as cells of the immune system generated in the bone marrow and involved in the humoral response. As known in the art, B cells can be identified by the presence of specific phenotypic biomarkers secreted by the cells. In the context of the invention, the term “B cells” designate a cell population wherein the cells have the phenotype CD45^{bright}, SSC^{low}, CD3⁻, CD14⁻, CD56⁻, as defined by conventional FACS analysis. Preferably, the term refers to B cells present in the blood circulation of the patient, also called circulating B cells.

In the context of the invention, the term “CD19⁺ B cells” refers to B cells as defined herein, i.e. cells which have the phenotype CD45^{bright}, SSC^{low}, CD3⁻, CD14⁻, CD56⁻; and which further have the phenotype CD19⁺, as defined by conventional FACS analysis. Preferably, the term refers to CD19⁺ B cells present in the blood circulation of the patient, also called circulating CD19⁺ B cells.

In the context of the invention, the term “CD20⁺ B cells” refers to B cells as defined herein, i.e. cells which have the phenotype CD45^{bright}, SSC^{low}, CD3⁻, CD14⁻, CD56⁻; and which further have the phenotype CD20⁺, as defined by conventional FACS analysis. Preferably, the term refers to CD20⁺ B cells present in the blood circulation of the patient, also called circulating CD20⁺ B cells.

In the context of the invention, the term “activated B cells” refers to B cells as defined herein, i.e. cells which have the phenotype CD45^{bright}, SSC^{low}, CD3⁻, CD14⁻, CD56⁻; and which further have the phenotype CD20⁺, CD19⁻, CD69⁻, as defined by conventional FACS analysis. Preferably, the term refers to activated B cells present in the blood circulation of the patient, also called circulating activated B cells.

In the context of the invention, the term “naive B cells” refers to B cells as defined herein, i.e. cells which have the phenotype CD45^{bright}, SSC^{low}, CD3⁻, CD14⁻, CD56⁻; and which further have the phenotype CD20⁺, CD19⁺, IgD⁺, CD27⁻ as defined by conventional FACS analysis. Preferably, the term refers to naive B cells present in the blood circulation of the patient, also called circulating naive B cells.

In the context of the invention, the term “memory B cells” refers to B cells as defined herein, i.e. cells which have the phenotype CD45^{bright}, SSC^{low}, CD3⁻, CD14⁻, CD56⁻; and which further have the phenotype CD20⁺, CD19⁺, CD27⁺ as defined by conventional FACS analysis. Preferably, the term refers to memory B cells present in the blood circulation of the patient, also called circulating memory B cells.

In the context of the invention, the term “CD19⁺ memory B cells” refers to B cells as defined herein, i.e. cells which have the phenotype CD45^{bright}, SSC^{low}, CD3⁻, CD14⁻, CD56⁻; and which further have the phenotype CD19⁺ as defined by conventional FACS analysis. Preferably, the term refers to CD19⁺ memory B cells present in the blood circulation of the patient, also called circulating CD19⁺ memory B cells.

In the context of the invention, the term “short lived plasma cells” refers to B cells as defined herein, i.e. cells which have the phenotype CD45^{bright}, SSC^{low}, CD3⁻, CD14⁻, CD56⁻; and which further have the phenotype CD19^{dim}, CD20−/^{dim}, CD27^{bright} as defined by conventional FACS analysis. Preferably, the term refers to short lived plasma cells present in the blood circulation of the patient, also called circulating short lived plasma cells.

In the context of the invention, the term “Breg” or “Breg cells” refers to B cells as defined herein, i.e. cells which have the phenotype CD45^{bright}, SSC^{low}, CD3⁻, CD14⁻, CD56⁻; and which further have the phenotype CD19⁺, CD24^{bright}, CD38^{bright} as defined by conventional FACS analysis. Preferably, the term refer to Breg cells present in the blood circulation of the patient, also called circulating Breg cells.

In the context of the invention, the term “CD38^{bright} plasma cells” refers to B cells as defined herein, i.e. cells which have the phenotype CD45^{bright}, SSC^{low}, CD3⁻, CD14⁻, CD56⁻; and which further have the phenotype CD19^{dim}, CD20⁻, CD38^{bright} as defined by conventional FACS analysis. Preferably, the term refer to CD38^{bright} plasma cells present in the blood circulation of the patient, also called circulating CD38^{bright} plasma cells.

In the context of the invention, the term “Transitional B cells” refers to B cells as defined herein, i.e. cells which have the phenotype CD45^{bright}, SSC^{low}, CD3⁻, CD14⁻, CD56⁻; and which further have the phenotype CD19⁺, CD20⁺, IgD⁺, CD10⁺, CD27⁻ as defined by conventional FACS analysis. Methods to identify circulating human transitional B cells are well known in the art and have in particular been detailed by Sims et al. (Identification and characterization of circulating human transitional B cells. Blood. 2005; 105(11):4390-4398. doi:10.1182/blood-2004-11-4284). Preferably, the term refers to Transitional B cells present in the blood circulation of the patient, also called circulating Transitional B cells.

In the context of the invention, the term “CD4 naïve” or “CD4 naïve T cells” refer to lymphocyte which has the phenotype CD3⁺, CD8⁻, CD4⁺, CD45RA⁺, CCR7⁺ as defined by conventional FACS analysis. Preferably, the term refers to CD4 naïve present in the blood circulation of the patient, also called circulating CD4 naïve T cells.

In the context of the invention, the term “CD4 central memory” or “CD4 central memory T cells” refer to lymphocyte which has the phenotype CD3⁺, CD8⁻, CD4⁺, CD45RA⁻, CCR7⁺ as defined by conventional FACS analysis. Preferably, the term refers to CD4 central memory T cells present in the blood circulation of the patient, also called circulating CD4 central memory T cells.

In the context of the invention, the term “CD4 effector memory” or “CD4 effector memory T cells” refer to lymphocyte which has the phenotype CD3⁺, CD8⁻, CD4⁺, CD45RA⁻, CCR7⁺ as defined by conventional FACS analysis. Preferably, the term refers to CD4 effector memory T cells present in the blood circulation of the patient, also called circulating CD4 effector memory T cells.

In the context of the invention, the term “CD8 naive” or “CD8 naive T cell” refer to lymphocyte which has the phenotype CD3⁺, CD4⁻, CD8⁺, CD45RA⁺, CCR7⁺ as defined by conventional FACS analysis. Preferably, the term refers to CD8 naive T cells present in the blood circulation of the patient, also called circulating CD8 naive T cells.

In the context of the invention, the term “CD8 central memory” or “CD8 central memory T cell” refer to lymphocyte which has the phenotype CD3⁺, CD4⁻, CD8⁺, CD45RA⁻, CCR7⁺ as defined by conventional FACS analysis. Preferably, the term refers to CD8 central memory T cells present in the blood circulation of the patient, also called circulating CD8 central memory T cells.

In the context of the invention, the term “CD8 effector memory” or “CD8 effector memory T cell” refer to lymphocyte which has the phenotype CD3⁺, CD4⁻, CD8⁺, CD45RA⁻, CCR7⁻ as defined by conventional FACS analysis. Preferably, the term refers to CD8 effector memory T cells present in the blood circulation of the patient, also called circulating CD8 effector memory T cells.

In the context of the invention, the term “CD8 TEMRA” or “CD8 TEMRA T cell” refer to lymphocyte which has the phenotype CD3⁺, CD4⁻, CD8⁺, CD45RA⁺, CCR7⁻ as defined by conventional FACS analysis. Preferably, the term refers to CD8 TEMRA T cells present in the blood circulation of the patient, also called circulating CD8 TEMRA T cells.

In the context of the invention, the term “Th1” or “Th1 cells” refer to lymphocyte which has the phenotype CD3⁺, CD8⁻, CD4⁺, CCR7^{-/+}, CXCR3⁺ as defined by conventional FACS analysis. Preferably, the term refers to Th1 cells present in the blood circulation of the patient, also called circulating Th1 cells.

In the context of the invention, the term “Treg” or “Treg cell” refer to lymphocyte which has the phenotype CD3⁺, CD8⁻, CD4⁺, CD25^{bright}, CD127^{dim/-} as defined by conventional FACS analysis. Preferably, the term refers to Treg cells present in the blood circulation of the patient, also called circulating Treg cells.

In the context of the invention, the term “Th17” or “Th17 cell” refer to lymphocyte which has the phenotype CD3⁺, CD8⁻, CD4⁺, CD45RA⁻, CCR7^{-/dim}, CCR6⁺, CD146⁺ as defined by conventional FACS analysis. Preferably, the term refers to Th17 cells present in the blood circulation of the patient, also called circulating Th17 cells.

In the context of the invention, the term “Th2” or “Th2 cell” refer to lymphocyte which has the phenotype CD3⁺, CD8⁻, CD4⁺, CXCR3⁻, CCR6⁻ as defined by conventional FACS analysis. Preferably, the term refers to Th2 cells present in the blood circulation of the patient, also called circulating Th2 cells.

In the context of the invention, the term “NKp46 NK cells” refer to lymphocyte which has the phenotype SSC^{low}, CD45⁺, CD19⁻, CD3⁻, CD16⁺CD56⁺, CD335⁺ as defined by conventional FACS analysis. Preferably, the term refers to NKp46 NK cells present in the blood circulation of the patient, also called circulating NKp46 NK cells.

In the context of the invention, the term “CD16 NK cells” refer to lymphocyte which has the phenotype SSC^{low}, CD45⁺, CD19⁻, CD3⁻, CD16⁺CD56⁺ as defined by conventional FACS analysis. Preferably, the term refers to CD16 NK cells present in the blood circulation of the patient, also called circulating CD16 NK cells.

In the context of the invention, the term “CD^{bright} CD56^{dim} NK cells” refer to lymphocyte which has the phenotype SSC^{low}, CD45⁺, CD19⁻, CD3⁻, CD56^{dim}, CD16^{bright} as defined by conventional FACS analysis. Preferably, the term refers to CD16_{bright} CD56^{dim} NK cells present in the blood circulation of the patient, also called circulating CD16^{bright} CD56^{dim} NK cells.

In the context of the invention, the term “CD16d⁻CD56^{bright} NK cells” refer to lymphocyte which has the phenotype SSC^{low}, CD45⁺, CD19⁻, CD3⁻, CD56^{bright}, CD16^{-/+} as defined by conventional FACS analysis. Preferably, the term refers to CD16^{dim} CD56^{bright} NK cells present in the blood circulation of the patient, also called circulating CD16^{dim} CD56^{bright} NK cells.

In the context of the invention, the term “CD16⁻CD56^{dim} NK cells” refer to lymphocyte which has the phenotype SSC^{low}, CD45⁻, CD19⁻, CD3⁻, CD56^{dim}, CD16⁻ as defined by conventional FACS analysis. Preferably, the term refers to CD16⁻ CD56^{dim} NK cells present in the blood circulation of the patient, also called circulating CD16⁻CD56^{dim} NK cells.

In the context of the invention, the term “CD16⁺ CD56⁻ NK cells” refer to lymphocyte which has the phenotype SSC^{low}, CD45⁺, CD19⁻, CD3⁻, CD56⁻, CD16⁻ as defined by conventional FACS analysis. Preferably, the term refers to CD16⁺ CD56⁻ NK cells present in the blood circulation of the patient, also called circulating CD16⁺ CD56⁻ NK cells.

In the context of the invention, the term “CD16⁺ CD56⁺ NK cells” refer to lymphocyte which has the phenotype SSC^{low}, CD45⁺, CD19⁻, CD3⁻, CD56⁺, CD16⁺⁻ as defined by conventional FACS analysis. Preferably, the term refers to CD16⁺ CD56⁺ NK cells present in the blood circulation of the patient, also called circulating CD16⁺ CD56⁺ NK cells.

### B. Biomarkers (Immune Signatures) Supporting Cladribine Retreatment Decisions or DMD Switch

Current therapies of autoimmune diseases, preferably autoimmune neurological diseases and neuroinflammatory diseases, suffer from the inability to determine whether a patient is suitable to a certain treatment or treatment schedule, including the decision when to treat and when to treat with which therapeutic or medicament. Thus, there is a high medical need to determine, preferably predetermine or predict whether a patient's immune system is responding (or not) appropriately to a specific therapeutic, medicament, treatment and/or treatment schedule. In this context, there is also a high need for biomarkers which enable the prediction of disease activation or disease reactivation, which would allow the clinician to make a sound decision whether to treat/retreat or not, and if yes, when to treat, when to retreat and/or employing which therapeutic or medicament. The present invention preferably addresses one or more of these medical needs.

Preferably, compositions and/or methods are provided for individuals suffering from an autoimmune disease, preferably autoimmune neurological disease, preferably including one or more diseases selected from autoimmune demyelinating diseases, neuroinflammatory diseases, autoimmune muscular disorders, (RMS and PMS, Myasthenia Gravis (MG), Neuromyelitis Optica (NMO), Marie-Tooth Disease (CMT), Adrenoleukodystrophy (ALD), Acute Disseminated Encephalitis (ADEM), Giant Cell Arteritis, Multifocal Motor Neuropathy, Polymyositis, Diabetic Neuropathies).

More preferably, compositions and/or methods are provided for individuals suffering from an autoimmune disease, preferably autoimmune neurological disease, neuroinflammatory diseases, preferably including one or more diseases selected from autoimmune demyelinating diseases. Autoimmune neurological diseases and/or autoimmune demyelinating diseases preferably include multiple sclerosis (MS) and/or multiple sclerosis spectrum disorders (MSSD), neuromyelitis optica and/or neuromyelitis optica spectrum disorders (NMOSD), Myasthenia Gravis (MG), Marie-Tooth Disease (CMT), Adrenoleukodystrophy (ALD), Acute Disseminated Encephalitis (ADEM), Giant Cell Arteritis, Multifocal Motor Neuropathy, Polymyositis, Diabetic Neuropathies)

Compositions and/or methods are provided for individuals suffering from MS (or other demyelinating diseases and AI Neurology indication) to be divided into groups that are informative and/or predictive of the individuals'
responsiveness to treatment, preferably responsiveness to treatment with one or more DMDs (Disease Modifying Drugs), and especially responsiveness to treatment with cladribine;
lack of responsiveness to (future) treatment, preferably lack of responsiveness to (future) treatment with one or more DMDs (Disease Modifying Drugs), and especially lack of responsiveness to (future) treatment with cladribine;
loss of response to (previous) treatment, preferably loss of response to (previous) treatment with one or more DMDs (Disease Modifying Drugs), and especially loss of response to (previous) treatment with cladribine; and/or
premature or early loss of response to (previous) treatment, preferably or early premature loss of response to (previous) treatment with one or more DMDs (Disease Modifying Drugs), and especially premature or early loss of response to (previous) treatment with cladribine.
Loss of response and disease reactivation after long term remission in year 3 and 4 after cladribine treatment

In particular it is preferably shown that the effective immune modulatory treatment effect of the respective therapeutic or medicament, preferably the effective immune modulatory treatment effect the respective MID and especially the effective immune modulatory treatment effect of cladribine, in the respective individual is mediated though specific patterns of sustained decrease and/or reconstitution of specific B cells, specific T cells and/or NK cell subtypes in an RMS population.

These specific B cell, T cell and/or and NK cell subtypes can be divided in either pathological subtypes with pro-inflammatory and disease inducing and worsening potential and regulatory subtypes with an anti-inflammatory phenotype, playing a role in immune regulation and tolerance induction in MS and other autoimmune diseases, especially autoimmune inflammatory diseases. Immune subtypes can be identified by methods known and understood in the art, preferably by specific cell surface markers known and understood in the art, for example using fluorescence-activated cell sorting (FACS) using specific CD markers, Mass cytometry (CyTOF, cytometry by time of flight), a variation of flow cytometry in which antibodies are labeled with heavy metal ion tags rather than fluorochromes, immune deconvolution methods using bioinformatics algorithms or other methods known and understood by the ones skilled in the art. Patients whole blood or PBMCs (Peripheral blood mononuclear cells) isolated from blood is used for the identification of the immune cell subsets.

The increase or decrease of immune subsets may be measured on an absolute level, as a percentage of total peripheral blood mononuclear cells, as a percent to B cells or T cells, for example CD19⁺ or CD20⁺ B cells, CD4⁺ or CD8⁺ T cells. In addition the increase may be measured in relative to heathy control, relative to the same patient prior to therapy (baseline value), relative to a predefined standard.

Surprisingly, in the context of the instant invention, it was found that single immune cells types or “immune signatures” comprising a combination of multiple immune subtypes preferably serves as a biomarker of
response to (future and/or or previous) treatment, preferably treatment as described herein;
(future) non response to treatment, preferably treatment as described herein; and/or
(early or premature, late) loss of efficacy, response and/or responsiveness to (previous and/or future) to treatment, preferably treatment as described herein.

For example, a response or lack of efficacy can be preferably monitored by measuring an increased or reduced numbers of specific immune subtypes respectively or by altered ratios of pathological vs regulatory B, T or NK subtypes, or increased or reduced depletion of said specific immune subtypes respectively, or by altered ratios of pathological vs regulatory B, T or NK subtypes to a predefined standard.

According to the instant invention, it is preferably shown that treatment of said autoimmune diseases with DMDs, preferably the treatment of MS with said DMDs and especially treatment of MS with Cladribine, in an individual
i) induces an early sustained decrease of memory B cells in said individual, preferably with the steepest or major decrease in Month 1 and/or Months 2, counted from the start of the respective DMD treatment, which decrease or reduced level is sustained for several months, preferably Months 1 to 12 and 24 counted from said start of the respective DMD treatment,
ii) induces a sustained increase of regulatory B cells in said individual, preferably a sustained increase of regulatory B cells over baseline level prior to the start of said treatment, wherein said sustained increase preferably is in Months 2 to 14 and especially in Months 3 to 12, counted from the start of the respective DMD treatment, and/or
iii) induces a sustained increase of transitional B cells in said individual, preferably a sustained increase of transitional B cells over baseline level prior to the start of said treatment, wherein said sustained increase preferably is in Months 2 to 14 and especially in Months 3 to 12, counted from the start of the respective DMD treatment. This characteristic pattern/immune signature preferably correlates with or indicates an onset of the durable efficacy response to said DMD treatment, with efficacy response preferably sets in in Month 2 or 3 after the second monthly treatment course of cladribine, and preferably stays for at least 12 months. Thus, this characteristic pattern/immune signature is sustained up to 12 and indicative for or predictive of a durative effect of treatment with said DMD in said individual. This characteristic pattern/immune signature preferably follows or is indicative of a prolonged (month 6-12) of DMD efficacy as measured by MRI.

According to the instant invention, it is preferably shown that treatment of said autoimmune diseases with DMDs, preferably the treatment of MS with said DMDs and especially treatment of MS with Cladribine, in an individual is showing a sustained decrease of plasmablasts/plasma cells (short lived plasmablasts and CD38⁺ plasma cells involved in the formation of AB production and compartmentalized inflammation) from month 2-12. Thus, this characteristic pattern is indicative for a prediction of a long-term response.

According to the instant invention, it is preferably shown that treatment of said autoimmune diseases with DMDs, preferably the treatment of MS with said DMDs and especially treatment of MS with Cladribine, in an individual is inducing a decreased ratio in plasma cells signatures identified by immune deconvolution methods at 96 weeks after first cladribine treatment vs untreated control in CLARITY phase 3, which correlates with Mill activity parameters, indicative of prediction of long term response at 96 weeks after cladribine.

According to the instant invention, it is preferably shown that treatment of said autoimmune diseases with DMDs, preferably the treatment of MS with said DMDs and especially treatment of MS with Cladribine, in an individual does not or not significantly affect the number of anti-inflammatory regulatory NK cells (CD16^{low} CD56^{bright}), but decreases proinflammatory NK effector cells (CD16^{bright}-CD56^{dim}) leading to an increased NKCD56^{bright}/NKCD56^{dim} NK cells ratio (0.8 to 1.4), preferably throughout months 3 to 12, preferably counted from the start of the respective DMD treatment. An approx. 1.5-2.5-fold increase in this ratio supports response to treatment.

According to the instant invention, it is preferably shown that treatment of said autoimmune diseases with DMDs, preferably the treatment of MS with said DMDs and especially treatment of MS with Cladribine, in an individual decreases CD4⁺ and CD8⁺ naïve, central and effector memory T cells with Nadir values at month 3 and 6 and limited repopulation at month 12 in the MAGNIFY and week 96 in the Mavenclad CLARITY trial. As a novel biomarker to monitor a durable response more than 10 month after the second year Cladribine treatment Naïve T cells show a significant positive correlation with MRI parameters using immune deconvolution signatures to identity T cells subtypes in the CLARITY 96 weeks.

According to the instant invention, it is preferably shown that treatment of said autoimmune diseases with DMDs, preferably the treatment of MS with said DMDs and especially treatment of MS with Cladribine, in an individual induces a sustained decrease of pathological memory B cells and plasmablast/cells (>−70% and >−50% from baseline respectively) from month 1-12 and month 3-12 respectively. Repopulation of these cells resulting in a increase of numbers of memory B cells and Plasma cells to <−40% and −20% respectively will illustrate a loss of efficacy serving as a retreatment marker. The invention therefore also pertains to a method of monitoring the response of a patient having an autoimmune disease, preferably chosen among autoimmune neurological and neuroinflammatory diseases, to treatment with a disease-modifying drug (DMD), preferably Cladribine, said method comprising:
a. obtaining the absolute numbers of any of:
   total PBMCs
   CD19⁺ B cells
   CD19⁺ memory B cells;
   naive B cells;
   CD4⁺ Central memory T cells;
   CD4⁺ Effector memory T cells;
   Treg;
   Teff;
   Breg;
   memory B cells;
   CD16^{dim} CD56^{bright} NK cells;
   CD16^{bright} CD56^{dim} INK cells;
   transitional B cells;
      in a first and a second biological sample from the patient,
wherein any of:
   an increase of the absolute number of CD19+ B cells or a decrease of 30% or less of the absolute number of CD19+ B cells, in the second biological sample compared to the first biological sample;
   an increase of the absolute number of CD19+ memory B cells or a decrease of 30% or less of the absolute number of CD19+ memory B cells, in the second biological sample compared to the first biological sample; and/or
   an increase of the absolute number of naive B cells or a decrease of 30% or less of the absolute number of naive B cells, in the second biological sample compared to the first biological sample;
   an increase of the absolute number of CD4+Central memory T cells or a decrease of 20% or less of the absolute number of CD4+Central memory T cells, in the second biological sample compared to the first biological sample;
   an increase of the absolute number of CD4⁺ Effector memory T cells or a decrease of 10% or less of the absolute number of CD4⁺ Effector memory T cells, in the second biological sample compared to the first biological sample;
   a decrease in the ratio of the absolute number of Treg over the absolute number of Teff in the second biological sample compared to said ratio in the first biological sample;
   a decrease in the ratio of the absolute number of Breg cells over the absolute number of memory B cells in the second biological sample compared to said ratio in the first biological sample;
   a decrease in the ratio of the absolute number of CD16^{dim} CD56^{bright} NK cells over the absolute number of CD16^{bright} CD56^{dim} NK cells in the second biological sample compared to said ratio in the first biological sample;
   a percentage of memory B cells over total PBMCs of between 0.05% and 1% in the second biological sample;
   a percentage of Breg cells over total PBMCs of between 3% and 14%, preferably of between 1% and 10% in the second biological sample;
   a ratio of Transitional B cells:memory B cells of superior or equal to 1, preferably comprised between 1 and 3, in the second biological sample;
is indicative of a sub-optimal response of the patient to the treatment.

Preferably, the first biological sample is a sample collected prior to the first administration or administration period with regard to the respective DMD.

Preferably, the second biological sample is a sample. collected after the start of the first administration of the treatment and (i) during the course of the treatment or (ii) after the last administration of the treatment. More preferably the second biological sample is a sample collected at least one months, preferably at least two months from the start of the first administration or administration period with regard to the respective DMD.

In an embodiment, the second biological sample is a sample collected within 1 to 12 months from the start of the first administration or administration period with regard to the respective DMD. In another embodiment, the second biological sample is a sample collected within 13 to 24 months from the start of the first administration or administration period with regard to the respective DMD.

In the context of the method of the invention, step a) of obtaining absolute numbers can be performed by obtaining data formerly generated by in vitro analysis of said first and second sample through a separate method, or be performed by determining said absolute numbers as active steps of the method.

The invention therefore also pertains to a method of monitoring the response of a patient having an autoimmune disease, preferably chosen among autoimmune neurological and neuroinflammatory diseases, to treatment with a disease-modifying drug (DMD), preferably Cladribine, said method comprising:
b. obtaining a first biological sample from the patient,
c. obtaining a second biological sample from the patient;
d. determining in the first and the second biological sample the absolute numbers of any of:
   total PBMCs
   CD19⁺ B cells
   CD19⁺ memory B cells;
   naive B cells;
   CD4⁺ Central memory T cells;
   CD4⁺ Effector memory T cells;
   Treg;
   Teff;
   Breg;
   memory B cells;
   CD16^{dim} CD56^{bright} NK cells;
   CD16^{bright} CD56^{dim} NK cells;
   transitional B cells;
wherein any of:
   an increase of the absolute number of CD19+ B cells or a decrease of 30% or less of the absolute number of CD19+ B cells, in the second biological sample compared to the first biological sample;
   an increase of the absolute number of CD19+ memory B cells or a decrease of 30% or less of the absolute number of CD19+ memory B cells, in the second biological sample compared to the first biological sample; and/or
   an increase of the absolute number of naive B cells or a decrease of 30% or less of the absolute number of naive B cells, in the second biological sample compared to the first biological sample;
   an increase of the absolute number of CD4+Central memory T cells or a decrease of 20% or less of the absolute number of CD4+Central memory T cells, in the second biological sample compared to the first biological sample;
   an increase of the absolute number of CD4⁺ Effector memory T cells or a decrease of 10% or less of the absolute number of CD4⁺ Effector memory T cells, in the second biological sample compared to the first biological sample;
   a decrease in the ratio of the absolute number of Treg over the absolute number of Teff in the second biological sample compared to said ratio in the first biological sample;
   a decrease in the ratio of the absolute number of Breg cells over the absolute number of memory B cells in the second biological sample compared to said ratio in the first biological sample;
   a decrease in the ratio of the absolute number of CD16^{dim} CD56^{bright} NK cells over the absolute number of CD16^{bright} CD56^{dim} NK cells in the second biological sample compared to said ratio in the first biological sample;
   a percentage of memory B cells over total PBMCs of between 0.05% and 1% in the second biological sample;
   a percentage of Breg cells over total PBMCs of between 3% and 14%, preferably of between 1% and 10% in the second biological sample;
   a ratio of Transitional B cells:memory B cells of superior or equal to 1, preferably comprised between 1 and 3, in the second biological sample;
is indicative of a sub-optimal response of the patient to the treatment.

The method of monitoring the response of a patient according to the invention may be usefully integrated in the course of a treatment.

The invention therefore also pertains to a method of treating a patient having an autoimmune disease, preferably chosen among autoimmune neurological and neuroinflammatory diseases, to treatment with a disease-modifying drug (DMD), preferably Cladribine, said method comprising:
a. obtaining a first biological sample from the patient,
b. administering the treatment to the patient;
c. obtaining a second biological sample from the patient;
d. determining in the first and the second biological sample the absolute numbers of any of:
   total PBMCs
   CD19⁺ B cells
   CD19⁺ memory B cells;
   naive B cells;
   CD4⁺ Central memory T cells;
   CD4⁺ Effector memory T cells;
   Treg;
   Teff;
   Breg;
   memory B cells;
   CD16^{dim} CD56^{bright} NK cells;
   CD16^{bright} CD56^{dim} NK cells;
   transitional B cells;
wherein any of:
   an increase of the absolute number of CD19+ B cells or a decrease of 30% or less of the absolute number of CD19+ B cells, in the second biological sample compared to the first biological sample;
   an increase of the absolute number of CD19+ memory B cells or a decrease of 30% or less of the absolute number of CD19+ memory B cells, in the second biological sample compared to the first biological sample; and/or
   an increase of the absolute number of naive B cells or a decrease of 30% or less of the absolute number of naive B cells, in the second biological sample compared to the first biological sample;
   an increase of the absolute number of CD4+Central memory T cells or a decrease of 20% or less of the absolute number of CD4+Central memory T cells, in the second biological sample compared to the first biological sample;
   an increase of the absolute number of CD4⁺ Effector memory T cells or a decrease of 10% or less of the absolute number of CD4⁺ Effector memory T cells, in the second biological sample compared to the first biological sample;
   a decrease in the ratio of the absolute number of Treg over the absolute number of Teff in the second biological sample compared to said ratio in the first biological sample;
   a decrease in the ratio of the absolute number of Breg cells over the absolute number of memory B cells in the second biological sample compared to said ratio in the first biological sample;
   a decrease in the ratio of the absolute number of CD16^{dim} CD56_{bright} NK cells over the absolute number of CD16_{bright} CD56_{dim} NK cells in the second biological sample compared to said ratio in the first biological sample;
   a percentage of memory B cells over total PBMCs of between 0.05% and 1% in the second biological sample;
   a percentage of Breg cells over total PBMCs of between 3% and 14%, preferably of between 1% and 10% in the second biological sample;
   a ratio of Transitional B cells:memory B cells of superior or equal to 1, preferably comprised between 1 and 3, in the second biological sample;
is indicative of a sub-optimal response of the patient to the treatment.

In the context of the invention, the indication of a sub-optimal response of the patient to the treatment is indicative of a lack of efficacy of the treatment, such as for instance lack of short-term efficacy and/or long-term efficacy of treatment, and the lack of indication of a sub-optimal response of the patient to the treatment is indicative of efficacy of the treatment, such as for instance short-term efficacy and/or long-term efficacy of treatment.

Preferably, an indication of a lack of efficacy of the treatment as determined by the methods of the invention warrants additional administration periods with said DMD for said patients, a switch to another DMD, and/or the application of one or more additional treatment options, preferably within 1 to 12 months following said determination of said indication of lack of efficacy, and an indication of short-term efficacy does not require additional administration periods with said DMD for said patients, or a switch to another DMD or other treatment option. CD4 and CD8 (including CD4⁺TH1 and CD8⁺TH17 cells) at 3 or 6 month after the first dose of cladribine but cells recover at M12 to levels which is found to be indicative for a need for retreatment to reach full Cladribine efficacy in year.

Thus the method as described above may further comprise the step of administering to the patient with a sub-optimal response to the treatment a retreatment with cladribine, an additional and/or higher dose of cladribine or the administration of a different DMD.

In the context of the invention, a retreatment with cladribine preferably comprises or consist in the oral administration of a formulation comprising cladribine, wherein the formulation is to be orally administered following the sequential steps below:
(i) an induction period wherein said cladribine formulation is administered and wherein the total dose of cladribine reached at the end of the induction period is from about 1.7 mg/kg to about 3.5 mg/kg;
(ii) a cladribine-free period of between about 8 and about 10 months wherein no cladribine formulation is administered;
(iii) a maintenance period wherein said cladribine formulation is administered and wherein the total dose of cladribine reached at the end of the maintenance period is lower than the total dose of cladribine reached at the end of the induction period (i); and
(iv) a cladribine-free period wherein no cladribine formulation is administered.

According to the instant invention, it is preferably shown that treatment of said autoimmune diseases with DMDs, preferably the treatment of MS with said DMDs and especially treatment of MS with Cladribine, in an individual, does less affect CD4+ Tregs and CD8+ TEMRA cells in comparison to CD4+ and CD8+ effector cells, suggesting a increased Treg/Teff ratio suggestive of tolerance induction involved in long term efficacy.

C. Use of Cladribine in Specific MS Population and Other AI/Neuroinflammatory Diseases with Plasmablast/Cell Involvement

MS is a heterogenous disease and specific populations respond less well to treatment. Current DMDs do not target Plasmablasts and Plasma cell subtypes, indicative of more severe MS disease, directly and efficiently (Tellesford et al 2020). The present invention addresses this need.

According to the instant invention, it is preferably shown that treatment of treatment of MS with Cladribine, in an individual, reduces short lived plasmablasts and CD38⁺ plasma cells and the direct effect on these plasma cell subpopulations is further supported by blood data from MS patients vaccinated twice with the Shingrix vaccine before the start of treatment with the DMD Cladribine. After the second Shingrix dose given 1 week before said start of cladribine treatment, the patient raised an acute plasma cell response, and increased plasma cell numbers in the blood to the MS patient were reduced by cladribine treatment (cite vaccination patent). These findings indicate that Cladribine is able to directly affect and reduce plasmablasts and CD38+ plasma cells which are associated with disease pathology by means of autoantibody production and direct cellular toxicity, especially when occurring within the cerebrospinal fluid. What we found in MS is also important for other diseases. There are a variety of AI & Neuroinflammatory diseases where potentially plasma cells/plasmablasts are involved in the disease activity and disease course which we deem to benefit from a treatment directly acting on plasma cells, preferably cladribine treatment. The list of preferred diseases with said involvement of plasmablasts and potential use of Cladribine comprises, but is not limited to Multifocal Motor Neuropathy (MMN), Chronic Inflammatory Demyelinating Polyneuropathy (CIDP), Guillain-Barre Syndrome (GBS), Inclusion Body Myositis (IBM), Myasthenia Gravis (MG), and/or Neuromyelitis Optica (NMO). Furthermore, the list of diseases with potential involvement of plasmablasts and therefore also potential use of Cladribine comprises but is not limited to Giant Cell arteritis (GCA), Polymoysitis, Microscopic Polyangiitis (MPA), Amyotrophic Lateral Sclerosis (ALS), and/or CNS Vasculitis (CNSV).

We found further evidence that in many diseases where B cells and autoantibodies play a role, Plasmablasts/cells are a valuable treatment target, as theses cell populations have been implicated with autoantibody production, and cladribine will be the DMD of choice due its direct depletion effect on short lived and CD38+ plasma cells. The list of preferred diseases with involvement of auto-antibodies and therefore potential use of Cladribine comprises but is not limited to Acute Disseminated Encephalitis (ADEM), Giant Cell Arteritis (GCA), Multifocal Motor Neuropathy (MMN), Polymyositis, Adrenoleukodystrophy (ALD), Anti-MAG Peripheral Neuropathy, Charcot-Marie-Tooth Disease (CMT), Diabetic Neuropathies, Frontotemporal Dementia (FTD), Guillain-Barre Syndrome (GBS), Hashimoto's Encephalopathy (HE), Graves' disease (GD), Huntington's Disease (HD), Inclusion Body Myositis (IBM), Microscopic Polyangiitis (MPA), Myasthenia Gravis (MG), and/or Neuromyelitis Optica (NMO). Furthermore, the list of diseases with involvement of B-cells or for which B-cell depleting therapies have shown a therapeutic and therefore also potential use of Cladribine comprises but is not limited to preferably Giant Cell arteritis (GCA), Multifocal Motor Neuropathy (MMN), Polymoysitis, Chronic Inflammatory Demyelinating Polyneuropathy (CIDP), Anti-MAG Peripheral Neuropathy, Inclusion Body Myositis (IBM), Microscopic Polyangiitis (MPA), Myasthenia Gravis (MG), Neuromyelitis Optica (NMO), Neurosarcoidosis, and/or CNS Vasculitis (CNSV). The invention therefore also pertains to a method of decreasing short term plasmablasts and/or CD38+ plasmablasts in a patient in need thereof, preferably a patient having an autoimmune disease, said method comprising administering a disease-modifying drug (DMD), preferably Cladribine, to said patient. In other terms, the invention also pertains to a disease-modifying drug (DMD), preferably Cladribine, for use in decreasing short term plasmablasts and/or CD38+ plasmablasts in a patient in need thereof, preferably a patient having an autoimmune disease.

In the context of the invention, a patient in need of decreasing short term plasmablasts and/or CD38+ plasmablasts is a patient having an autoimmune disease, yet preferably an autoimmune disease chosen in the list consisting in multiple sclerosis (MS) and/or multiple sclerosis spectrum disorders (MSSD), neuromyelitis optica and/or neuromyelitis optica spectrum disorders (NMOSD), Myasthenia Gravis (MG), Marie-Tooth Disease (CMT), Adrenoleukodystrophy (ALD), Acute Disseminated Encephalitis (ADEM), Giant Cell Arteritis, Multifocal Motor Neuropathy, Polymyositis, Diabetic Neuropathies, Frontotemporal Dementia (FTD), Guillain-Barre Syndrome (GBS), Hashimoto's Encephalopathy (HE), Graves' disease (GD), Huntington's Disease (HD), Inclusion Body Myositis (IBM), Microscopic Polyangiitis (MPA)

A preferred aspect of the instant invention is the newly found role of pathological and regulatory B and T cells subtypes in MS and other AI/Neuroinflammatory diseases, preferably inflammatory and/or demyelinating autoimmune diseases, and especially MS:

MS is considered a chronic inflammatory demyelinating disease of the CNS, leading to the loss of myelin, along with axonal and neuronal degeneration. Historically, the factors considered to induce MS include the activation of pathogenic Th1, Th17, and CD8+ myelin auto-reactive T cells, in addition to genetics, and environmental factors, and infections.

The immune response feedback is believed to be an important mechanism that maintains the immune balance. Many Inflammatory diseases including, but not limited to multiple sclerosis (MS) are hallmarks of immunologic imbalances. As a major component of the immune system, B cells play both positive and negative roles in innate and adaptive immunity, through effector molecules such as antibodies and cytokines as well as through antigen-presentation. On the one hand, B cells can mediate several potentially negative processes, such as amplifying immune responses. Mechanistically, they differentiate into plasmablasts that secrete effector antibodies, and may modulate effector T cell response through antigen presentation and/or production of inflammatory cytokines. In addition, there is also a subset of B cells that regulates immune response to pathogens and autoantigens. These regulatory B cells are core targets for therapeutic treatment in autoimmune and infectious diseases as well as cancer.

Recently, anti-CD20 therapies targeting B cells and B cell cytokines (rituximab, ocrelizumab, ofatumumab, and tabalumab), and cladribine, selectively targeting B cells and T cells, have been shown to suppress MS disease activity. Furthermore, B cell depletion in MS can reduce auto-antibodies and pro-inflammatory cytokines produced by B cells and impaired antigen presentation. B cell and plasmablast/cell infiltration was found in the brain tissues of patients with MS, and oligoclonal bands (clonal IgG) and antibody-secreting plasma cells have been discovered in the cerebrospinal fluid. Meningeal ectopic B cell follicles are involved in progression and appear to be increased in secondary-progressive MS. B cells are centrally involved in the pathogenesis of autoimmunity, exerting diverse effects such as contributing to T cell activation through antibody production and antigen presentation. Antibody secreting cells (ASCs) represent a continuum of antigen-activated B cells that, through differentiation into plasmablasts and mature plasma cells, dedicate metabolic and protein processing mechanisms to antibody production. Indeed, plasmablasts and plasma cells, as ASCs derived from antigen-experienced B cells, appear to be important drivers of both inflammatory and neurodegenerative aspects of MS pathogenesis, and preferably other inflammatory and/or demyelinating autoimmune disorders. ASCs are enriched within the CSF during active gadolinium-enhancing disease, ASC-derived intrathecal IgG correlates with CNS atrophy, and IgM-producing ASCs are associated with aggressive disease course. Despite heterogeneity across the ASC spectrum, these cells may be collectively identified as brightly positive for CD27 and CD38.

B and T cells may exert multiple pro-inflammatory actions, but also possess regulatory functions making their role in the pathogenesis of MS and other AI diseases much more complex. Besides antigen presenting and antibody production, B cells are found to also play a role in immune regulation and tolerance induction through IL-10-dependent and -independent mechanisms. A variety of B-cell subsets have been documented as regulatory B cells (Bregs), with CD19+CD24hiCD38hi expression B cells and/or (CD45^{bright}, SSClow, CD3−, CD14−, CD56−, CD19+, CD24bright, CD38bright have been identified as the principal IL-10-expressing subsets (“bright” and “hi” are preferably used as synonyms, if not explicitly specified otherwise). Bregs are usually induced and maintained in response to inflammation and participate in immunomodulations and in suppression of an increased immune responses. The regulatory effects of Bregs were described in various models of inflammation, autoimmune diseases, transplantation reactions and in anti-tumor immunity. However, it was not until the late 1990s that two independent studies showed that autoimmune diseases (experimental autoimmune encephalomyelitis (EAE) and chronic colitis) deteriorated in the B-cell-deficient group, providing further evidence of Bregs and suggesting their function in suppressing inflammation. Bregs regulate the immune system by different mechanisms. The main mechanism is a production of anti-inflammatory cytokine interleukin 10 (IL-10). IL-10 has strong anti-inflammatory effects. and it inhibits or suppresses inflammatory reactions mediated by T cells, especially Th1 or TH17 type immune reactions. This was shown for example in models of MS (EAE, Experimental Encephalomyelitis), RA, CIA (collagen induced arthritis) or contact hypersensitivity.

Another suppressive Breg mechanisms include the production of transforming growth factor (TGF-β) and the regulation of cell surface molecules, including FasL and/or PD-L1, which cause death of target cells.

Transitional B cells (TrB cells) are bone marrow-derived, immature B cells, which are also considered to be precursors of mature B cells. TrB cells represent one of the regulatory B cell subpopulations in healthy individuals, but the frequency of CD24^{hi}CD38^{hi} TrB cells in circulation may be altered in individuals with autoimmune diseases, such as multiple sclerosis, neuromyelitis optica spectrum disorders and systemic lupus erythematosus. TrB cells can produce IL-10 and regulate CD4+ T cell proliferation and differentiation toward T helper (Th) effector cells. According to our current understanding, the main functions of human CD19⁺CD24^{hi}CD38^{hi} TrB cells at this time are: first, IL-10 produced by TrB cells suppresses autoreactive CD4⁺ T cell proliferation. Second, TrB cells suppress the production of pro-inflammatory cytokines by limiting the expansion of CD4+ Th1 cells (IFN-γ and TNF-α production) and CD4+Th17 cells (IL-17 production), which is dependent on IL-10, programmed cell death-ligand 1 (PD-L1), CD80, and CD86, but not on transforming growth factor-β (TGF-β). Third, TrB cells prevent the CD4+ T cells from differentiating into Th1 and Th17 cells and promote the conversion of effector CD4+ T cells into CD4+FoxP3+ Tregs while limiting the production of excessive pro-inflammatory cytokines. Finally, TrB cells inhibit CD8⁺ T cell responses and maintain invariant nature killer T (iNKT) cells.

Furthermore, significantly fewer TrB cells were detected in individuals or patients, preferably body fluid samples of individuals or patients, with MS/clinically isolated syndrome (CIS), compared to healthy controls. Similar to that of TrB cells, the frequency of other circulating B-cell subsets (naïve, MZ-like, and memory B cells) in peripheral blood of MS/CIS cases were comparable to those in healthy controls. NMOSDs are idiopathic inflammatory demyelinating diseases affecting the CNS, and are clinically characterized by longitudinally extensive transverse myelitis (LETM) and severe optic neuritis (ON), traditionally considered a variant of Asian MS. Up to now, only a few studies have been conducted to explore the roles of TrB cells in NMOSDs. Specifically, fewer CD19+CD24hiCD38hi TrB cells were found in individuals or patients, preferably body fluid samples of individuals or patients, with NMOSDs than in those with MS. The frequencies of CD19+CD27+ memory B cells and mature B cells are not significantly different between those with NMOSDs and HC, whereas the percentage of CD19+CD5+CD1dhi Bregs decreased in NMOSDs cases. The proportion of TrB cells has been demonstrated to be lower in AQP4-positive group than in AQP4-negative group. Moreover, the frequencies of IL-10-expressing B cells among all lymphocytes are reported to be lower in individuals or patients, preferably body fluid samples of individuals or patients, with NMOSDs than in those with MS and in healthy individuals. This indirectly shows the impairment of immunomodulatory function of TrB cells in NMOSDs due to a reduction of TrB cells and/or IL-10.

Thus the administration of a disease-modifying drug (DMD), in particular Cladribine, to subjects presenting with an autoimmune disease characterized by an alteration of the number of TrB cells in the blood circulation, an in particular by a decrease of the number of TrB cells in blood circulation compared to healthy subjects, in particular multiple sclerosis (MS) provides an increase in Transitional B cells and/or Regulatory B cells.

Another aspect of the invention is therefore the use of disease-modifying drug (DMD), preferably Cladribine, for modulating immune tolerance in a patient, preferably for improving immune tolerance in a patient in need thereof.

The invention further pertains to a method for increasing Transitional B cells and/or Regulatory B cells in a patient in need thereof, preferably a patient having an autoimmune disease, said method comprising administering a disease-modifying drug (DMD), preferably Cladribine, to said patient. The invention pertains to a method for improving immune tolerance, preferably by increasing Transitional B cells and/or Regulatory B cells, in a patient in need thereof, preferably a patient afflicted with an autoimmune disease, said method comprising administering a disease-modifying drug (DMD), preferably Cladribine, to said patient.

In the context of the invention, a patient in need of increasing Transitional B cells and/or Regulatory B cells is a patient having an autoimmune disease as defined herein, preferably chosen in the list consisting in multiple sclerosis (MS) and/or multiple sclerosis spectrum disorders (MSSD), and neuromyelitis optica and/or neuromyelitis optica spectrum disorders (NMOSD).

Tregs were originally identified by Sakaguchi et al. in 1995 as a CD4+CD25+ T cell subset with suppressive activity. They are essential to the maintenance of self-tolerance and their impairment has been linked with autoimmunity and includes numerical decreases, functional defects and conversion into inflammatory effector cells. High expression of CD25 and low expression of CD127 are the main phenotypic markers characterizing bona fide human Tregs. The discovery of forkhead box P3 (FoxP3), as a fundamental transcription factor for the development of regulatory CD4+CD25+ T cells in the thymus, helped researchers to precisely phenotype most Tregs. Currently, Tregs may be accurately identified as CD4+CD25+FoxP3+ T cells or (as FoxP3 inversely correlate with cell surface CD127 expression) as CD4+CD25+CD127lo/− T cells. Tregs have been found to play a pivotal function in regulating the immune system by controlling the number and function of effector cells. Thus, they play a major role in suppressing unwanted autoreactive immune responses, such as in the case of autoimmunity. Interestingly, it has been indicated that Tregs can modulate both adaptive and innate immune systems, and once activated they specifically regulate immune responses at multiple levels and by various mechanisms. These suppressive mechanisms can be organized into major groups, including cell-cell contact-dependent suppression, inhibitory cytokine release (such as IL-10 and TGF-β), modulation of APC function, cytolysis, metabolic disruption and induction of suppressor cells or “infectious tolerance”. Recent studies suggest that the innate immune system plays an important role in both the initiation and progression of MS by influencing the effector function of T and B cells.

Natural killer (NK) cells are found to contribute to both effector and regulatory functions of innate immunity via their cytotoxic activity and their ability to secrete pro- and anti-inflammatory cytokines and growth factors. Mechanisms by which NK cells could have an impact on autoimmune responses include a rapid cytokine release before autoreactive T cell differentiation and modulation of interactions between autoreactive T and B lymphocytes and antigen-presenting cells. Several NK subsets responsible for different functions have been identified. Based on the surface expression of CD56, NK cells have been classified as effector (CD56dim) or regulatory (CD56bright) cells. Effector NK cells are characterized by intracellular expression of perforin and granzymes, which are proteolytic enzymes involved in target killing. They may be subdivided further into CD56+CD16+(cytotoxic effector cells) and CD56+CD3+(NK T cells), which may produce a variety of cytokines and control other immune cells. Conversely, CD56bright NK regulatory cells represent fewer than 10% of peripheral blood NK cells, express low levels of perforin and are able to secrete large amounts of cytokines. In addition, CD56−CD16+ NK cells are a defective NK subset with impaired cytolytic function that increases in viraemic HIV and hepatitis C virus (HCV)-infected individuals. It has been suggested that NK cells play a key role at the interface of innate and adaptive responses in autoimmune diseases. Depending on the cell subtype and milieu, both pathogenic and protective roles in the central nervous system (CNS) have been attributed to different NK subpopulations in MS and its animal model, experimental autoimmune encephalomyelitis (EAE). In keeping with this, NK cell lines may induce lysis of oligodendrocytes. In addition, CNS-resident NK cells may inhibit T helper type 17 (Th17) differentiation in EAE by interacting with microglia cells. MS patients are found to show a significant increase of regulatory/effector (CD56bright/CD56dim) NK ratio compared to other neurological diseases (IND). The bright/dim ratio in the CSF remained higher in active MS [1.0 (0.57-1.89), median (25-75% interquartile range)] when compared to IND patients [0.43 (0.25-1.28), P=0.019]. Different studies have described changes in the percentages of NK cells in the CSF of MS patients. Moreover, the beneficial effect of daclizumab, a humanized monoclonal antibody (mAb) that blocks the binding site on the IL-2Rα chain (CD25), is linked to the expansion of the CD56bright cells. Expansion of regulatory CD56bright NK cells associated with a good response to daclizumab and IFN-0, while CD56dim cells decreased after immunomodulatory therapy.

### Transitional B Cells and B Regs

B cells are centrally involved in the pathogenesis of autoimmunity, exerting diverse effects such as contributing to T cell activation through antibody production and antigen presentation. B cells also regulate immunological functions by suppressing T cell proliferation and producing pro-inflammatory cytokines, such as interferon-gamma (IFN-γ), tumor necrosis factor-α (TNF-α) and interleukin (IL)-17.

B cells develop from pre-B cells through immature cells in the BM to transitional cells in the periphery and then to peripheral naïve cells in a linear pathway. Transitional B cells (TrB cells) represent a crucial link between immature B cells in the bone marrow and mature peripheral B cells. Although TrB cells represent one of the regulatory B cell subpopulations in healthy individuals, the frequency of CD24^{hi}CD38^{hi} TrB cells in circulation may be altered in individuals with autoimmune diseases, such as multiple sclerosis, neuromyelitis optica spectrum disorders, systemic lupus erythematosus, Sjögren's syndrome, rheumatoid arthritis, systemic sclerosis, and juvenile dermatomyositis. Although TrB cells play regulatory roles under inflammatory conditions, consequences of their functional impairment vary across autoimmune diseases.

The process of B cell development in humans is similar to that in mice.

(B cells originating from HSCs can be processed in the bone marrow, where they undergo heavy and light chain rearrangements before entering the peripheral compartment. HSCs initially stem from common lymphoid progenitors and then develop into pro-B cells via rearrangement of the D and J segments following juxtaposition of a variable (VH) gene with the DJH element. Pro-B cells mature into pre-B cells via the rearrangement of μ-H-chain gene segments, which couple with a surrogate light chain. After initial rearrangement of the light chain (V-L), human pre-B cells undergo 1 or 2 cell divisions and develop into immature B cells, which first express μ/κ or κ/λ, surface IgM receptors. These immature T1 B cells are thought to emigrate from the bone marrow, subsequently differentiating into T2 B cells in the periphery, eventually giving rise to T3 B cells. However, there is some disagreement regarding this process. TrB cells might be capable of departing from the bone marrow at either the T1 or T2 B cell stage, and some T1 B cells can differentiate into T2 B cells in the bone marrow, considering that similar proportions of T1 and T2 B cell subsets has been noted in normal human bone marrow. In agreement with previous findings in mice, positive and negative selection may also regulate the development of human TrB cells. In normal human peripheral blood, approximately 40% of TrB cells show autoreactive behavior; the frequency is reduced to 20% when they are differentiated into mature naïve B cells. TrB cell maturation requires Btk, demonstrating a possibility of BCR engagement, and is accompanied by improved survival. BAFF promotes immature B cell selection and non-autoreactive immature B cells differentiation into TrB cells, although the role of BAFF in human TrB cell development has been controversial. Some previous data had shown BAFF to have no effect on human TrB cells, although it serves as an efficient pro-survival factor for mouse TrB cells. Some data has indicated that human T1 B cell proliferation to be clearly induced by BAFF, though to a lesser extent than that of human T2 B cells. Therefore, during the process of TrB cell differentiation into mature naïve B cells, selection

Transitional B cells (TrB cells) are bone marrow-derived, immature B cells, which are also considered to be precursors of mature B cells. TrB cells account for approximately 4% of all CD19+ B lymphocytes in healthy individuals. They are present in human bone marrow, peripheral blood, cord blood, and secondary lymphoid tissues (i.e., spleen, tonsil, lymph nodes, and gut-associated lymphoid tissue [GALT]).

CD24hiCD38hi TrB cells are closely related to IL-10-producing regulatory B cells (Bregs) in terms of phenotypical and functional similarities. TrB cells can also produce IL-10 and regulate CD4+ T cell proliferation and differentiation toward T helper (Th) effector cells. A low frequency of TrB cells has been noted in those with neuroimmunological diseases, including multiple sclerosis (MS) and neuromyelitis optica spectrum disorders (NMOSDs). Since they are associated with several inflammatory diseases and are also found in circulation as well as tissues of healthy individuals, TrB cells are thought to perform distinct functions in immune-defense mechanisms.

### Transitional B Cells in Humans and Relationship to Bregs

Human TrB cells were first described in detail in 2005, and are often characterized by a CD24hiCD38hi phenotype. Human CD24hiCD38hi TrB cells represent approximately 4% of all CD19+ B cells in the peripheral blood, and constitute nearly 50% of B cells in cord blood and their proportion gradually declining during infancy. Human TrB cells have also been found in GALT. Human TrB cells were discriminated into three subsets, including T1 B cells, T2 B cells, and T3 B cells.

In 2016, Simon first proposed human CD24hiCD38hi TrB cells to not contain only one subset, but rather 4 subsets based on CD27, IgM, IgD, CD10, CD21, and CD32 expression. T1-T3 B cells express low levels of CD27, whereas CD27+ TrB cells express CD27, CD24, and CD38 at high levels. In T1 B cells, the expression of IgM, CD10, and CD32 is high while that of IgD and CD21 is low. T2 B cells show moderate IgM, IgD, CD10, and CD32 expression and low CD21 expression. However, T3 B cells express IgM, IgD, CD10, CD21, and CD32 at low levels.

Some studies indicated that CD27+ TrB cells serve as a type of Bregs and may not just being the transitional B cells. Bregs contribute to the maintenance of immune tolerance and modulation of immune responses. Currently, human Bregs have been noted at different stages of B cell development and consist of various B cell subsets, namely CD24hiCD38hi immature TrB cells, CD24hiCD38loCD27+ memory B cells, and CD24−CD38+CD27int IL-10-secreting plasmablasts. Human IL-10-producing Bregs are enriched within the CD24hiCD38hi immature TrB cell subset. These observations reveal a relationship between human TrB cells and Bregs.

### The Main Functions of Transitional B Cells

Human TrB cells produce low amounts of Ig and exhibit less differentiation, proliferation, and chemotaxis in vitro than mature B cells. Human IL-10+ B cells with a transitional CD19+CD24hiCD38hi phenotype have been found in peripheral blood of healthy individuals. The main functions of human CD19+CD24hiCD38hi TrB cells discovered at this time have been summarized in FIG. 2. First, IL-10 produced by TrB cells suppresses autoreactive CD4+ T cell proliferation. Second, TrB cells suppress the production of pro-inflammatory cytokines by limiting the expansion of CD4+ Th1 cells (IFN-γ and TNF-α production) and CD4+Th17 cells (IL-17 production), which is dependent on IL-10, programmed cell death-ligand 1 (PD-L1), CD80, and CD86, but not on transforming growth factor-β (TGF-β). Third, TrB cells prevent the CD4+ T cells from differentiating into Th1 and Th17 cells and promote the conversion of effector CD4+ T cells into CD4+FoxP3+ Tregs while limiting the production of excessive pro-inflammatory cytokines. Finally, TrB cells inhibit CD8+ T cell responses and maintain invariant nature killer T (iNKT) cells.

Recent findings have revealed different subpopulations of TrB cells to have different functions. For example, T2/T3 B cells have a significant capacity to decrease CD4+ T cell proliferation (contrary to other subpopulations), and only CD27+ TrB cells significantly reduced TNF-α and IFN-7 production by CD4+ T cells. In addition to producing anti-inflammatory factors, TrB cells can also secrete pro-inflammatory cytokines such as IL-6 and TNF-α. Data from previous studies had shown the frequency of IL-6-producing TrB cells to be elevated in SLE, SS, and other immune diseases. IL-6 restricts the differentiation of CD4+ T cells into Tregs and induces autoreactive Th1/Th17 T-cell responses. Therefore, the pro- and anti-inflammatory cytokines produced in TrB cells may affect their functional stability.
1) TrB cells suppress the proliferation of autoreactive CD4+ T cells.
2) TrB cells prevent the differentiation of CD4+ T cells into Th1 and Th17 cells, thus limiting the production of excessive pro-inflammatory cytokines (TNF-α, IFN-γ, and IL-17).
3) TrB cells promote the conversion of effector CD4+ T cells into CD4+FoxP3+ Tregs.
4) TrB cells inhibit CD8+ T cell responses. The suppressive functions of TrB cells are partially dependent on the secretion of IL-10, which can down-regulate CD86 expression in an autocrine manner. TrB cells can also secrete pro-inflammatory cytokines, such as IL-6 and TNF-α. The imbalance between pro- and anti-inflammatory cytokine productions in TrB cells may affect their functional stability and thus participate in the development of immune diseases.

### TrB Cells and TrB-Associated Molecules in MS and EAE

MS is considered a chronic inflammatory demyelinating disease of the CNS, leading to the loss of myelin, along with axonal and neuronal degeneration. The pathologic features of MS are typically characterized by the dissemination of time and space. Historically, the factors considered to induce MS include the activation of pathogenic Th1, Th17, and CD8+ myelin auto-reactive T cells, in addition to genetics, and environmental factors, and infections.

In support of this animal model, EAE also can be induced by myelin-specific CD4+ T cells and CD8+ T cells. Recently, therapies targeting B cells and B cell cytokines (rituximab, ocrelizumab, ofatumumab, and tabalumab) have been shown to suppress MS disease activity. Furthermore, B cell depletion in MS can reduce auto-antibodies and pro-inflammatory cytokines produced by B cells and impair antigen presentation. B cell infiltration was found in the brain tissues of patients with MS, and oligoclonal bands (clonal IgG) and antibody-secreting plasma cells have been discovered in the cerebrospinal fluid. Meningeal ectopic B cell follicles were formatted in secondary-progressive MS. Collectively, these observations demonstrated the participation of B cells in the pathogenesis of MS. Recent research has shown B cells to be virtually depleted after anti-CD20 treatment in EAE, then after 6-8 weeks, they gradually reappeared in the bone marrow, spleen, lymph nodes, and blood. Surprisingly, splenic TrB and follicular B cells were found to be almost absent in EAE treated with anti-CD20, at 8 weeks post-treatment.

In addition to their involvement in MS pathogenesis, B cells also exert immune-regulatory effects by producing anti-inflammatory cytokines directly or indirectly. Bregs play a regulatory role in MS by helping with the maintenance of immune tolerance. In mice, IL-10-producing CD1dhiCD5+ B cells play a more substantial role in suppressing EAE initiation and development than other B cells. TrB cells account for a considerable proportion of functional Breg cells. Significantly fewer TrB cells were detected in those with MS/clinically isolated syndrome (CIS). Similar to that of TrB cells, the frequency of other circulating B-cell subsets (naïve, MZ-like, and memory B cells) in peripheral blood of MS/CIS cases were comparable to those in healthy controls.

Some data further demonstrate TrB cells to be capable of homing to inflamed sites in the CNS through up-regulated expression of α4 and β1 integrins during the early phase of MS. TrB cells in the peripheral blood of those with MS who were treated with fingolimod, produced more IL-10 and less TNF-α than memory and mature naïve B cells, and expressed low levels of CD80. Related studies have shown the immune-regulatory role of Bregs to be partly mediated through the production of IL-10, IL-35, TGF-β and PD-L1. In EAE, CD40 signaling is required for inducing IL-10 competence. Subsequently, IL-21 drives the expansion of IL-10-producing B cell as well as generation of effector cells. IL-6-producing B cells are involved in the pathogenesis of EAE/MS. Over 65% of IL-6 is primarily produced by B cells, which exacerbates EAE, augments the autoreactive T cell responses of Th1 and Th17 types, and inhibits the generation of Tregs. TrB cells, like Bregs, play an immune-regulatory role in MS/EAE

### TrB Cells and TrB-Associated Molecules in Neuromyelitis Optica Spectrum Disorders

NMOSDs are idiopathic inflammatory demyelinating diseases affecting the CNS, and are clinically characterized by longitudinally extensive transverse myelitis (LETM) and severe optic neuritis (ON), traditionally considered a variant of Asian MS. Lennon et al. first discovered a serological marker of neuromyelitis optica (NMO), known as the aquaporin-4 antibody (AQP4-Ab) or NMO-IgG, making NMO independent of MS. The identification of AQP4-Ab broadened the spectrum of NMO. AQP4-Ab is involved in the pathogenesis of NMOSDs, depending on the presence of B cells, T cells, and complement, suggesting NMOSDs to possibly be primarily mediated by humoral immunity. NMOSDs cases can be associated with other autoimmune diseases such as SLE, SS, and thyroiditis, implying that NMOSDs may involve a mechanism resembling that of autoimmune diseases. Like SLE and other autoantibody-mediated immune diseases, NMOSDs cases have deficiencies in the integrity of the central and peripheral B cell tolerance checkpoints. In addition, impaired peripheral B cell tolerance checkpoints interfere with the removal of autoreactive B cells, leading to increase the reservoir self-reactive new emigrant/transitional and mature naive B cells, and this process has relation with the production of pathogenic anti-AQP4 autoantibody.

Currently, only a few studies have been conducted to explore the roles of TrB cells and TrB-associated molecules in NMOSDs. Specifically, fewer CD19+CD24hiCD38hi TrB cells were found in those with NMOSDs than in those with MS. The frequencies of CD19+CD27+ memory B cells and mature B cells are not significantly different between those with NMOSDs and HC, whereas the percentage of CD19+CD5+CD1dhi Bregs decreased in NMOSDs cases. The proportion of TrB cells has been demonstrated to be lower in AQP4-positive group than in AQP4-negative group. Moreover, the frequencies of IL-10-expressing B cells among all lymphocytes are reported to be lower in those with NMOSDs than in those with MS and in healthy individuals. This indirectly shows the impairment of immunomodulatory function of TrB cells in NMOSDs due to a reduction of TrB cells and/or IL-10; IL-10 production in TrB cells was not measured in most studies. Rituximab treatment in those with NMOSDs lowered the number of CD27+ memory B cells and increased the number of CD24hiCD38hi TrB cells. Related studies also showed elevation of BAFF, IL-6, and IL-21, both in serum and CSF of NMOSDs cases, although expression of these molecules in TrB cells still remains unclear. AQP4-Ab is mainly derived from a subtype of B cells, CD27hiCD38hiCD180− B cells, survival of which depends on signaling via the IL-6 receptor, thus indicating IL-6-dependent B cells to possibly be associated with NMOSDs pathogenesis. Korn and colleagues had provided evidence of IL-6 and IL-21 suppressing Tregs production, despite inducing IL-17 secretion from CD4+ Th17 cells. Further studies would be required to explore the mechanism driving the IL-6 production of TrB cells in those with NMOSDs. If the proportion of IL-6-producing TrB cells is elevated in NMOSDs cases, then IL-6 secretion may impair the immune-regulatory function of TrB cells.

Characterization of peripheral immune cell dynamics and repopulation patterns of cladribine treatment, preferably in the first 12 months of cladribine treatment:

Markers of early efficacy under and/or after DMD treatment and preferably under and/or after cladribine treatment (e.g. in Months 1 and/or 2):

% Decrease from Baseline
Decrease of CD19⁺ B cells (>70%),
Decrease of memory CD19⁺B cells (>70%), and/or
Decrease of both, Naïve and Memory B cells (>70%);
and/or combinations thereof.

Markers of long term efficacy under and/or after DMD treatment and preferably under and/or after cladribine treatment (e.g. Months 3-6-12, and preferably beyond, e.g. up to 4 year, up to 6 years, up to 8 years or up to 10 years, and preferably beyond):

% Decrease from Baseline
Sustained decrease of memory CD19⁺B cells (>70%) and/or plasma cells (>50%),
Increased levels of Bregs and transitional B cells (>70%), and/or
Decrease of naïve T cells, T central memory and effector cells (TBD);
and/or combinations thereof.

Markers of durable efficacy under and/or after DMD and preferably under and/or after cladribine treatment: Regulatory/effector cell ratio preferably increase or decrease from baseline, if applicable
Increased NKreg/NKeff ratio, compared to baseline, preferably >1.5 fold compared to baseline,
Increased Breg/Beff cells ratio compared to baseline, and/or
Increased Treg/Teff cell ratio) compared to baseline;
and/or combinations thereof

Markers of nonresponse under and/or after DMD treatment and preferably under and/or after cladribine treatment, e.g. in Months 1 and/or 2%

% Decrease from Baseline
CD19+ B cell decrease <30%,
memory CD19⁺B cell decrease <30%, and/or
Naïve <30%;
and/or combinations thereof.

Markers of disease reactivation under and/or after DMD treatment and preferably under and/or after cladribine treatment:

% Deviation from Baseline
Memory B cells repopulating to a level above −40% from baseline and/or plasma cells repopulating to a level above −20% from baseline
Tₙₐᵢᵥₑ, T_{CM} and/or T_{EM} repopulating to a level above −40%, −20%, and −10%, respectively,
Decreased Treg/Teff ratio and/or Breg/Bmem compared to baseline,
Decreased NKreg/NK eff ratio compared to baseline;
and/or combinations thereof.

Preferred characterization of peripheral immune cell dynamics and repopulation patterns of cladribine treatment, preferably in the first 12 months of cladribine treatment:

Preferred markers of early efficacy under and/or after DMD treatment and preferably under and/or after cladribine treatment (e.g. in Months 1 and/or 2):

% Decrease from Baseline
Decrease of CD19⁺ B cells (>70%),
Decrease of memory CD19⁺B cells (>70%), and/or
Decrease of both, Naïve and Memory B cells (>70%);
Decrease of activated B cells (CD69+; >45%)
and/or combinations thereof.

Preferred markers of long term efficacy under and/or after DMD treatment and preferably under and/or after cladribine treatment (e.g. Months 3-6-12, and preferably beyond, e.g. up to 4 year, up to 6 years, up to 8 years or up to 10 years, and preferably beyond)

% Decrease from Baseline
Sustained decrease of memory CD19⁺B cells (>70%) and/or plasma cells (>50%),
Increased levels of Bregs and transitional B cells (>70%), and/or
Decrease of naïve T cells, T central memory and effector cells (>10%, >40%, >30%);
Decrease of TH1 and RH17 cells (>35% each)
and/or combinations thereof.

Preferred markers of durable efficacy under and/or after DMD and preferably under and/or after cladribine treatment: Regulatory/effector cell ratio, % of whole blood or PBMC preferably increase or decrease from baseline, if applicable
Increased NKreg/NKeff ratio, compared to baseline, preferably >1.5 fold compared to baseline,
Increased Breg/Beff cells ratio compared to baseline, and/or
Increased Treg/Teff cell ratio) compared to baseline;
% Breg stable MS (preferable 14-20%, even more preferable 10-30%)
and/or combinations thereof

Markers of nonresponse under and/or after DMD treatment and preferably under and/or after cladribine treatment, e.g. in Months 1 and/or 2%

% Decrease from Baseline
CD19+ B cell decrease <30%,
memory CD19⁺B cell decrease <30%, and/or
Naïve<30%;
and/or combinations thereof.

Preferred markers of disease reactivation under and/or after DMD treatment and preferably under and/or after cladribine treatment
Memory B cells increase (preferable to 0.05 and 1% of total PBMCs) in combination with increased Transitional B cell ratio in total PBMC
% of Bregs in whole blood from active or reactivating MS patients (preferable 3-14%, even more preferable 1-10%)
Increase of Transitional B cells to Memory B cells (ratio of absolute cell counts: preferable >1, more preferable 1-3)

Preferred markers of disease reactivation under and/or after DMD treatment and preferably under and/or after cladribine treatment:

% Deviation from Baseline
Memory B cells repopulating to a level above −40% from baseline and/or plasma cells repopulating to a level above −20% from baseline
Tₙₐᵢᵥₑ, T_{CM} and/or T_{EM} repopulating to a level above −40%, −20%, and −10%, respectively,
Decreased Treg/Teff ratio and/or Breg/Bmem compared to baseline,
Decreased NKreg/NK eff ratio compared to baseline;
and/or combinations thereof.

Preferably, further quantitative examples of biomarker-active cells and/or cell populations are given below:

| Quantitative | | | Breg/total | |
|---|---|---|---|---|
| Characterization of changes | | NKreg/NKeff | CD19⁺ B cells | Treg/Teff |
| 1. Ratio | “Markers of durable | Increased | Increased | Increased |
| change from | efficacy under and/or | NKreg/NKeff ratio, | Breg/Beff cells | Treg/Teff cell |
| baseline | after DMD and preferably | compared to | ratio compared | ratio compared |
| (relative) | under and/or after | baseline, preferably > | to baseline, | to baseline, . . . |
| | cladribine treatment: | 1.5 fold compared | . . . and/or | (Based on |
| | Regulatory/effector | to baseline, | (Based on | MAGNIFY data) |
| | cell ratio preferably | (Based on | MAGNIFY data) | |
| | increase or decrease | MAGNIFY data) | | |
| | from baseline, if | | | |
| | applicable” | | | |
| Data n2. | “Markers of durable | NKreg/NKeff ratio | Breg/total CD19⁺ | Treg/Th17 ratio |
| Ratio change | efficacy under and/or | further increased | B cell ratio | increased to 1.3, |
| from | after DMD and preferably | compared to untreated | increased to 2.2%, | preferably to 2.5, |
| baseline | under and/or after | population (1.0) | preferably to 2.7%, | even more |
| (absolute) | cladribine treatment: | | even more | preferably to 3.6 |
| | Regulatory/effector | | preferably to 3.2% | from baseline |
| | cell ratio preferably | | from baseline | |
| | increase or decrease | | | |
| | from baseline, if | | | |
| | applicable” | | | |
| 3. Reg cells | “Markers of durable | Nkreg cells count | Breg cells count | Treg cells count |
| change | efficacy under and/or | further increased | increased to | increased to |
| from | after DMD and preferably | compared to untreated | 4.5 × 10³/ml, | 5.7 × 10³/mm³, |
| baseline | under and/or after | population (14.66 | preferably to | preferably to |
| (absolute) | cladribine treatment: | cells/ml) | 6.0 × 10³/ml, | 7.1 × 10³/mm³, |
| | Regulatory cell | | even more | even more |
| | absolute count preferably | | preferably to | preferably to |
| | increase or decrease | | 7.5 × 10³/ml | 9.0 × 10³/mm³ |
| | from baseline, if | | from baseline | from baseline |
| | applicable” | | | |

| % of Bregs in | “Markers of durable efficacy | % Breg stable MS (preferable 14-20 |
|---|---|---|
| whole blood | (stable MS) or remission (active | %, even more preferable 10-30%) |
| of stable vs | MS) under and/or after DMD and | and active MS (preferable 3-14%, |
| active MS | preferably under and/or after | even more preferable 1-10%) |
| | cladribine treatment: % Breg of | |
| | whole blood | |

Thus in an aspect, the invention pertains to a method to predict the response of a patient treated with cladribine for the treatment of an autoimmune disorder, preferably multiple sclerosis,
a) wherein deviations of cell populations of:
   CD19⁺B cells of at least 70%,
   memory CD19⁺B cells of at least 70%,
   naïve B cells of at least 70%,
   memory B cells of at least 70%,
   activated B cells, and/or
   activated CD69+ B cells of at least 45%,
and/or combinations thereof;
are indicative of early efficacy of said cladribine treatment;
wherein:
   cell populations are determined in one or more body fluids samples of said patient at different stages of said cladribine treatment, preferably including determining said cell population in a body fluids sample from the patient collected before the start of treatment with cladribine and determining said cell population in a body fluids sample from the patient collected at at least a later point in time during or after treatment with cladribine,
   said deviations are given as decrease from the baseline levels;
b) wherein deviations of cell populations of:
   CD19⁺B cells of at least 70%,
   CD38+ Plasmacells of at least 50%,
   Short lived plasmacells of at least 50%,
   Bregs of at least 70%,
   transitional B cells of at least 70%,
   naïve T cells of at least 10%,
   T central memory cells of at least 40%,
   T central effector cells of at least 30%,
   TH1 cells of at least 35%,
   TH17 cells of at least 35%,
and/or combinations thereof;
are indicative of durable efficacy of said cladribine treatment at 1 year
wherein:
   cell populations are determined in one or more body fluids samples of said patient at different stages of said cladribine treatment, preferably including determining said cell population in a body fluids sample from the patient collected before the start of treatment with cladribine and determining said cell population in a body fluids sample from the patient collected at at least a later point in time during or after treatment with cladribine,
   said deviations are given as decrease from the baseline levels;
c) wherein:
   a deviation of cell populations of memory B cells to a level above −40%,
   a deviation of cell populations of plasma cells to a level above −20%,
   a deviation of cell populations of Tnaive cells to a level above −40%,
   a deviation of cell populations of CD4+ Central memory T cells to a level above −20%,
   a deviation of cell populations of CD4+ Effector Memory T cells to a level above −10%
   a deviation of cell populations of the Treg/Teff cell ratio wherein the deviation is a decrease,
   a deviation of cell populations of the Breg/B memory cell ratio wherein the deviation is a decrease,
   a deviation of cell populations of the NKreg/NK eff cell ratio wherein the deviation is a decrease,
   and/or combinations thereof,
are indicative of a risk of reactivation of multiple sclerosis
wherein:
   cell populations are determined in one or more body fluids samples of said patient at different stages of said cladribine treatment, preferably including determining said cell population in a body fluids sample from the patient collected before the start of treatment with cladribine and determining said cell population in a body fluids sample from the patient collected at at least a later point in time during or after treatment with cladribine,
   said deviations are from the baseline levels.

Thus in an aspect, the invention pertains to a method for monitoring the need for adapting the treatment of an autoimmune disorder, preferably multiple sclerosis, with a Desease Modifying Drug (DMD) of a patient in need thereof,
wherein the patient has been treated with a DMD which is cladribine for the treatment of an autoimmune disorder, preferably multiple sclerosis,
wherein deviations of cell populations as determined in one or more body fluids samples of said patient at different stages of said cladribine treatment, preferably including determining said cell population in a body fluids sample from the patient collected before the start of treatment with cladribine and determining said cell population in a body fluids sample from the patient collected at at least a later point in time during or after treatment with cladribine are selected from the list consisting in:
a deviation of cell populations of memory B cells to a level above −40%,
a deviation of cell populations of plasma cells to a level above −20%,
a deviation of cell populations of Tnaive cells to a level above −40%,
a deviation of cell populations of CD4+ Central memory T cells to a level above −20%,
a deviation of cell populations of CD4+ Effector Memory T cells to a level above −10%
a deviation of cell populations of the Treg/Teff cell ratio wherein the deviation is a decrease,
a deviation of cell populations of the Breg/B memory cell ratio wherein the deviation is a decrease,
a deviation of cell populations of the NKreg/NK eff cell ratio wherein the deviation is a decrease,
and/or combinations thereof;
   wherein said deviations are indicative of a need for a retreatment of said patient with cladribine.

Thus, in an aspect, the invention pertains to method for the treatment of an autoimmune disorder, preferably multiple sclerosis, with a Disease Modifying Drug (DMD) of a patient at risk of reactivation of multiple sclerosis,
wherein the patient has been treated with a DMD which is cladribine for the treatment of an autoimmune disorder, preferably multiple sclerosis,
wherein deviations of cell populations as determined in one or more body fluids samples of said patient at different stages of said cladribine treatment, preferably including determining said cell population in a body fluids sample from the patient collected before the start of treatment with cladribine and determining said cell population in a body fluids sample from the patient collected at at least a later point in time during or after treatment with cladribine are selected from the list consisting in:
a deviation of cell populations of memory B cells to a level above −40%,
a deviation of cell populations of plasma cells to a level above −20%,
a deviation of cell populations of Tnaive cells to a level above −40%,
a deviation of cell populations of CD4+ Central memory T cells to a level above −20%,
a deviation of cell populations of CD4+ Effector Memory T cells to a level above −10%
a deviation of cell populations of the Treg/Teff cell ratio wherein the deviation is a decrease,
a deviation of cell populations of the Breg/B memory cell ratio wherein the deviation is a decrease,
a deviation of cell populations of the NKreg/NK eff cell ratio wherein the deviation is a decrease,
and/or combinations thereof;
   are indicative that the patient is a patient at risk of reactivation of multiple sclerosis,
   wherein said treatment comprises administering to the patient a retreatment with cladribine, the administration of an additional and/or higher dose of cladribine or the administration of a different DMD.

Thus, in one aspect, preferred is method of treating an autoimmune disorder in a patient in need thereof, said method preferably comprising orally administering cladribine to said patient, preferably administering cladribine orally to said patient in an amount of 0.5 to 1.25 mg per kilogram of body weight of said patient, preferably administering cladribine orally to said patient in an amount of 0.5 to 1.25 mg per kilogram of body weight of said patient in a time period between within 4 to 12 weeks, wherein, after said administration, said patient is having one or more deviations with regard to one or more of the cell populations, selected from the group consisting of:
a) wherein the following deviations are biomarkers/correspond to early efficacy of said cladribine treatment, given as % decrease from the baseline levels)
   CD19⁺ B cells (>70%, preferably +/−10%),
   memory CD19⁺B cells (>70%, preferably +/−10%)
   naïve B cells (>70%, preferably +/−10%)
   memory B cells (>70%, preferably +/−10%)
   activated B cells, and/or
   activated CD69+ B cells (>45, preferably +/−10%),
and/or combinations thereof;
b) Durable efficacy at 1 year (% decrease from baseline)
   memory CD19⁺B cells (>70%, preferably +/−10%),
   CD38+ Plasmacells (>50%, preferably +/−10%)
   Short lived plasmacells (>50%, preferably +/−10%)
   Bregs (>70%, preferably +/−10%),
   transitional B cells (>70%, preferably +/−10%),
   naïve T cells (>10%, preferably +/−5%),
   T central memory cells (>40%, preferably +/−10%),
   T central effector cells (>30%, preferably +/−10%),
   TH1 cells (>35, preferably +/−10%)—TH17 cells (>35, preferably +/−10%),
and/or combinations thereof;
c) Durable efficacy
   a NKreg/NKeff cell ratio (>1.5 fold),
   a Breg/Beff cell ratio (>/<fold),
   a Treg/Teff cell ratio (>/<fold), and/or
d) Reactivation of disease
   decrease of Memory B cells from baseline (<30%, preferably +/−10%)
   memory B cells (preferably to 0.05 and 1% of PBMCs),
   a transitional B cell ratio (>/<????), preferably in total PBMC,
   Bregs, preferably in whole blood or PBMCs, preferably from active or reactivating MS patients (preferable 3-14%%, even more preferable 1-10%), and/or
e) Reactivation of Disease
   memory B cells (preferably repopulating to a level above −40% from baseline),
   plasma cells (preferably repopulating to a level above −20% from baseline),
   Tₙₐᵢᵥₑ cells (preferably repopulating to a level above −40% from baseline,
   CD4+ Central memory T cells (preferably repopulating to a level above −20% from baseline,
   CD4+ Effector Memory T cells (preferably repopulating to a level above −10% from baseline,
   a Treg/Teff cell ratio (preferably decreased from baseline), and/or
   a Breg/B memory cell ratio (preferably decreased compared to baseline), and/or
   a NKreg/NK eff cell ratio (preferably decreased compared to baseline;
   and/or combinations thereof.

In the EU and the plurality of more than 80 countries around the world, MAVENCLAD® (cladribine tablets) is indicated for the treatment of adult patients with relapsing multiple sclerosis (MS), preferably highly active relapsing multiple sclerosis (MS) as defined by clinical or imaging features. The recommended cumulative dose of MAVENCLAD® is 3.5 mg/kg body weight over 2 years, administered as 1 treatment course of 1.75 mg/kg per year.

In the US, MAVENCLAD® (cladribine tablets) MAVENCLAD® (cladribine) tablets is indicated for the treatment of relapsing forms of multiple sclerosis (MS), to include relapsing-remitting disease and active secondary progressive disease, in adults. MAVENCLAD is administered in 2 treatment courses approximately 1 year apart, wherein the recommended cumulative dosage of MAVENCLAD is 3.5 mg/kg body weight, administered orally and divided into 2 yearly treatment courses (1.75 mg/kg per treatment course), preferably as follows:

### Year 1 Treatment Course:

First cycle (month 1): Start any time.
Second cycle (month 2): Start 23-27 days after the last dose (approximately 1 month after beginning of first cycle).

### Year 2 Treatment Course:

First cycle (month 1): Start approximately 1 year after beginning the first course, at least 43 weeks after the last dose.
Second cycle (month 2): Start 23-27 days after the last dose (approximately 1 month after beginning first cycle).

Each treatment cycle consists of 4 or 5 consecutive days; administer the cycle dosage as 1 or 2 tablets once daily over 4 or 5 consecutive days.

With regard to the “month(s)” (or “Month(s)”) and years in the context of timings and/or time periods described herein, those timings and/or time periods are preferably counted from the day on which said patient received the first dose of said DMD, preferably cladribine, more preferably oral cladribine or cladribine tablets (e.g. Mavenclad®), and more preferably counted from the day on which said patient received the first dose of said DMD, preferably cladribine, more preferably oral cladribine or cladribine tablets (e.g. Mavenclad®), in the respective treatment period of about 1 year, the respective treatment year or (yearly) treatment course.

The term “baseline” as used herein is known and understood in the art. In the context of the instant invention it preferably refers to state of the individual or patient before the first treatment with the respective DMD for which said biomarkers or cells used as biomarkers according to the invention shall be determined. Thus, the term baseline preferably reflects the “normal” status or level of the individual or patient and the respective cells later (i.e. after DMD treatment) to be used as biomarkers according to the invention, but in the absence of such a DMD treatment/a previous DMD treatment. If the individual already received a DMD beforehand, preferably a washout period of at least 1 to 10 months, preferably at least 1 to 4 months, is recommended in order to let said cells come to a normal, i.e. untreated level or sufficiently close to an untreated level.

The term “mg/kg” as used herein preferably means “milligram per kilogram bodyweight”.

More preferably, the term “mg/kg” as used herein and more preferably as used in connection with the fixed dose, in which cladribine is administered orally to a patient, that term “mg/kg” preferably means “milligram” of cladribine (orally) administered “per kilogram of body weight” of said patient.

The “total dose” or “cumulative dose” preferably refers to the total dose of Cladribine administered during the treatment, i.e. the dose reached at the end of the treatment that is calculated by adding the daily doses. For example, the total dose of Cladribine corresponding to a treatment of 0.7 mg/kg Cladribine per day during 5 days is 3.5 mg/kg or the total dose of Cladribine corresponding to a treatment of 0.35 mg/kg Cladribine per day during 5 days is 1.7 mg/kg.

“The total effective dose” or “cumulative effective dose” preferably refers to the bioavailable dose of Cladribine after a given administration period, i.e. the bioavailable dose reached at the end of the treatment that is calculated by adding the daily doses reduced by the bioavailability coefficient. For example, the total effective dose of Cladribine corresponding to a treatment of 0.7 mg/kg Cladribine per day during 5 days wherein the bioavailability of Cladribine is of about 40% is 1.4 mg/kg or the total effective dose of Cladribine corresponding to a treatment of 0.35 mg/kg Cladribine per day during 5 days wherein the bioavailability of Cladribine is of about 40% is 0.7 mg/kg.

Typically, the bioavailability of Cladribine or of a Cladribine formulation used in the context of this invention is from about 20% to about 90%, preferably from about 25% to about 80%, more preferably from about 30% to about 70%, even more preferably from about 35% to about 60% and especially from about 40% to about 50%, such as about 35%, about 40%, about 45%, about 50% or about 55%. Especially preferably the bioavailability from cladribine tablets is 45%+/−25%, more preferably 45%+/−20%, even more preferably 45%+/−15%, even more preferably 45%+/−10%, and especially 45%+/−5%, preferably based on the total content of the active ingredient cladribine (2-CdA) in the cladribine containing formulation, preferably cladribine containing oral formulation, to be preferably used in the methods and/or treatment schemes according to the invention.

The term “bioavailability” as used herein with regard to pharmaceutical dosage forms, including, but not limited to tablets, is known and understood in the art. Also, methods and/or procedures for the reliable determination of said bioavailability are known, understood and described in the art. Those known methods and/or procedures are typically in line with the requirements of Good Manufacturing Practice (GMP) and/or the requirements of Good Clinical Practice (GCP) to an extent as required by Health Authorities such as the FDA and/or EMA, as well as others.

However, bioavailability can be preferably assessed using the following methodology:

Bioavailability, conventionally defined as the fraction of the dose reaching the systemic circulation, has been assessed after oral administration of a single 10 mg tablet of Mavenclad and of 3 mg of cladribine (Leustatin 1 mg/mL) administered as a 1-hour IV infusion, in a randomized, two-period, two-sequence cross over study in patients with multiple sclerosis, where cladribine plasma concentration were measured using a validated LC/MS/MS method. Bioavailability has been estimated by means of nonlinear mixed effect modeling (software NONMEM version VI [higher versions would apply too]), by incorporating it in the form of a logit transform (to constrain it to be within the 0-1 range) as a parameter in the integrated pharmacokinetic model, developed in merging several studies using Mavenclad in various conditions (e.g. fed/fasted) and of varying duration (see R M Savic 2017 (DOI 10.1007/s40262-017-0516-6)). This modeling took into account several covariates, such as food intake in relation to drug administration. In addition to the parameter estimate, its precision and inter-subject variance were also included in the pharmaco-statistical model and calculated. Following sequential model development, refinement and validation, cladribine bioavailability following Mavenclad 10 mg tablets administration has been estimated at 45.6%, preferably at about 45.6%, more preferably with a precision of 7.03% and/or variance (on a logit scale) of 22.3%.

However, also other methods and methodologies can be successfully applied, e.g. as described in “Population Pharmacokinetics of Cladribine in Patients with Multiple Sclerosis”, Radojka M. Savic, Ana M. Novakovic, Marianne Ekblom, Alain Munafo & Mats O. Karlsson; Clinical Pharmacokinetics volume 56, pages 1245-1253(2017), and/or “The Clinical Pharmacology of Cladribine Tablets for the Treatment of Relapsing Multiple Sclerosis”, Robert Hermann, Mats O. Karlsson, Ana M. Novakovic, Nadia Terranova, Markus Fluck & Alain Munafo; Clinical Pharmacokinetics volume 58, pages 283-297(2019), which are hereby incorporated by reference in their entirety.

More specifically, e.g. for use according to the approved label for cladribine tablets (MAVENCLAD®), and taking into account a bioavailability for cladribine tablets of a about 45%, the recommended cumulative effective dose to be preferably reached in the body of the patient by the administration of cladribine tablets (MAVENCLAD®) is preferably about 1.58 mg/kg body weight (as achieved by administering cladribine tablets (MAVENCLAD®) in an amount of about 3.5 mg/kg body weight, based on a bioavailability of the administered tablets of about 45% as outlined before) over 2 years, preferably administered as 1 treatment course of 0.79 mg/kg body weight ((as achieved by administering cladribine tablets (MAVENCLAD®) in an amount of about 1.75 mg/kg body weight, based on a bioavailability of the administered tablets of about 45% as outlined before) per year. According to the approved label, each treatment course preferably consists of 2 treatment weeks, one preferably at the beginning of the first month and one preferably at the beginning of the second month of the respective treatment year. Each treatment week preferably consists of 4 or 5 days on which a patient preferably receives 10 mg or 20 mg (one or two tablets), preferably as a single daily dose, preferably depending on body weight.

The term “United States Prescribing Information”, abbreviated as “USPI”, is known and understood in the art. Preferably, the USPI sets out the health authority agreed usage, preferably the US Federal Drug Agency agreed usage, preferably more specifically, the US Federal Drug Agency marketing approved usage of a drug. It provides information on usage for healthcare professionals, and is an intrinsic part of the application for Marketing Authorisation (MA) of a new drug or medicine within the United States. Preferably, the equivalent of the USPI in the European Union is the Summary of Product Characteristics (SmPC).

Reference to the USPI in the context of the instant invention preferably refers to the USPI of oral cladribine or cladribine tablets, marketed under the tradename MAVENCLAD®, Mavenclad®, or just Mavenclad. Preferably, reference to the USPI in the context of the instant invention with regard to cladribine tablets means reference to the USPI of cladribine tablets/MAVENCLAD® as valid as of 2020. Preferably, the reference to the USPI of cladribine tablets/MAVENCLAD® includes all contents of that USPI except for the content with regard to and immunisations and/or vaccinations against infections, preferably immunisations vaccinations and/or infections as described herein in the context of the instant invention. Preferably, the instant invention provides methods of treatment that are preferably advantageously improved over the content of the USPI with regard to said immunisations and/or vaccinations against infections.

The term “European Summary of Product Characteristics”, abbreviated as “EU SmPC” or “SmPC”, is known and understood in the art. Preferably it refers to a document describing the properties and the officially approved conditions of use of a medicine. Summaries of product characteristics form the basis of information for healthcare professionals on how to use the medicine safely and effectively. More specifically, the SmPC preferably sets out the health authority agreed usage, preferably the European Health Authority (or Authorities) agreed usage, preferably more specifically, the European Medicines Agency (EMA) marketing approved usage of a drug. It provides information on usage for healthcare professionals, and is an intrinsic part of the application for Marketing Authorisation (MA) of a new drug or medicine within the European Union. Preferably, the equivalent of the SmPC in the United States of America is the “United States Prescribing Information” or abbreviated, “USPI”.

Reference to the EU SmPC, or preferably SmPC, in the context of the instant invention preferably refers to the EU SmPC, or preferably SmPC, of oral cladribine or cladribine tablets, marketed under the tradename MAVENCLAD®, Mavenclad®, or just Mavenclad. Preferably, reference to the EU SmPC, or preferably SmPC, in the context of the instant invention with regard to cladribine tablets means reference to the EU SmPC, or preferably SmPC, of cladribine tablets/MAVENCLAD® as valid as of 2020. Preferably, the reference to the EU SmPC, or preferably SmPC, of cladribine tablets/MAVENCLAD® includes all contents of that EU SmPC, or preferably SmPC, except for the content with regard to and immunisations and/or vaccinations against infections, preferably immunisations vaccinations and/or infections as described herein in the context of the instant invention. Preferably, the instant invention provides methods of treatment that are preferably advantageously improved over the content of the EU SmPC, or preferably SmPC, with regard to said immunisations and/or vaccinations against infections.

“A week” preferably refers to a period of time of or about 5, about 6 or about 7 days.

“A month” preferably refers to a period of time of or about 28, about 29, about 30 or about 31 days.

The term “treatment” is known and understood in the art. In specific contents, the term can preferably also mean or include the sequential succession of an “induction treatment” and at least a “maintenance treatment”. In this specific context, a treatment may comprise an “induction treatment” and about one or about two or about three maintenance treatments.

Typically, a treatment according to the invention is of about 2 years (about 24 months) or about 3 years (about 36 months) or about 4 years (about 48 months), preferably about two years.

Preferably, a treatment according to the invention is of about two years (about 24 months) or about three years (about 36 months) or about for years (about 48 months), preferably about two years (about 24 months). If the treatment according to the invention is of about two years (about 24 months), it preferably contains two treatment periods or to treatment courses. If the treatment according to the invention is of about three years (about 36 months) or about four years (about 48 months), it preferably contains three treatment periods/treatment courses or four treatment period/treatment courses.

Preferably, the treatment periods or treatment courses according to the invention comprise one month, two months or three months, wherein cladribine or preferably cladribine tablets are administered to a subject, preferably a subject in need thereof. Preferably, the treatment periods or treatment causes according to the invention comprise two months, wherein cladribine preferably cladribine tablets are administered to said subject. Especially preferably, each treatment course consists of two treatment weeks, one preferably at the beginning of the first month of said treatment course, and one week preferably at the beginning of the second month of said treatment course. Preferably, the same holds true for treatment periods, as those terms are preferably interchangeable in the context of the instant invention.

Preferably, each said treatment week comprises four or five days on which a subject receives cladribine, preferably cladribine tablets, preferably at a daily dose of 10 or 20 mg, depending on the body weight of the patient, in order to achieve the dosing of cladribine in mg/kg of body weight as described herein.

The term “about” as used herein with respect to numbers, figures, ranges and/or amounts is preferably meant to mean “circa” and/or “approximately”. The meaning of those terms is well known in the art and preferably includes a variance, deviation and/or variability of the respective number, figure, range and/or amount of plus/minus 15% and especially of plus/minus 10%.

In any case, the term “about” as used herein with respect to numbers, figures, ranges and/or amounts is preferably meant to mean “circa” and/or “approximately”. The meaning of those terms is well known in the art and preferably includes a variance, deviation and/or variability of the respective number, figure, range and/or amount of at least plus/minus 5%.

Preferably, any ranges mentioned herein below include all values and subvalues between the lowest and highest limit of this range.

The terms “disorder(s)” and “disease(s)” as used herein are well-known and understood in the art. In the context of the present invention they are preferably used as synonyms and thus are preferably interchangeable, if the context they are used herein does not strongly implicate otherwise.

In the medical context, including, but not limited to treatment regimens, dosing schedules and clinical trial designs, for convenience and/or ease of use by patients, medical staff and/or physicians, as well as reliability and/or reproducibility of results etc., the terms “week”/“a week”, “month”/“a month” and/or “year”/“a year” can used with slight deviations from the definitions of the Gregorian calendar. For example, in said medical context, a month is often referred to as 28 days, and a year is often referred to 48 weeks.

Thus, in the context of the instant invention, the term “week” or “a week” preferably refers to a period of time of about 5, about 6 or about 7 days, more preferably about 7 days.

In the medical context, the term “month” or “a month” preferably refers to a period of time of about 28, about 29, about 30 or about 31 days, more preferably about 28, about 30 or about 31 days.

In the medical context, the term “year” or “a year” preferably refers to a period of time of about 12 months or to a period of time of about 48, about 50, or about 52 weeks, more preferably 12 months, or about 48 or about 52 weeks.

### Cladribine (2-CdA)

2-CdA and its pharmacologically acceptable salts may be used in the practice of this invention.

Cladribine can be formulated in any pharmaceutical preparation suitable for oral administration. Representative oral formulations of 2-CdA are described in (WO 96/19230; WO 96/19229; U.S. Pat. Nos. 6,194,395; 5,506,214; WO 2004/087100; WO 2004/087101), the contents of which are incorporated herein by reference. Examples of ingredients for oral formulations are given below.

Processes for preparing 2-CdA are well known in the art. For example, the preparation of 2-CdA is described in (EP 173,059; WO 04/028462; WO 04/028462; U.S. Pat. No. 5,208,327; WO 00/64918) and Robins et al., J. Am. Chem. Soc., 1984, 106: 6379. Alternatively, pharmaceutical preparations of 2-CdA may be purchased from Bedford Laboratories, Bedford, Ohio.

Oral administration of Cladribine may be in capsule, tablet, oral suspension, or syrup form. The tablet or capsules may contain from about 3 to 500 mg of Cladribine. Preferably they may contain about 3 to about 10 mg of Cladribine, more preferably about 3, about 5 or about 10 mg of Cladribine. The capsules may be gelatin capsules and may contain, in addition to Cladribine in the quantity indicated above, a small quantity, for example less than 5% by weight, magnesium stearate or other excipient. Tablets may contain the foregoing amount of the compound and a binder, which may be a gelatin solution, a starch paste in water, polyvinyl polyvinyl alcohol in water, etc. with a typical sugar coating.

Preferably, the term “Cladribine” as used in the context of the instant invention, if not explicitly defined to be otherwise, preferably means “oral cladribine”, i.e. the active ingredient cladribine to be orally administered according to the invention. Preferably, the cladribine to be orally administered according to the invention is administered in the form of an oral dosage form, such as a capsule, e.g. a capsule filled with cladribine solution, a capsule filled with cladribine powder, a cladribine containing tablet and especially a cladribine-cyclodextrin complex containing tablet, as it is marketed under the name of Mavenclad®.

Especially preferably, said cladribine or oral cladribine for use according to the instant invention is to be orally administered as a tablet comprising 10 mg of Cladribine in the form of a mixture of Cladribine, 2-hydroxypropyl-β-cyclodextrin and Cladribine-2-hydroxypropyl-β-cyclodextrin-complexes, wherein the weight ratio of Cladribine to 2-hydroxypropyl-β-cyclodextrin is between about 1:10 to about 1:16, preferably between 1:13 to 1:15

A preferred oral dosage form in the form of the tablet is described directly below:

A preferred composition of cladribine for oral use according to the invention is a tablet containing 10 mg of cladribine (2-CdA), containing hydroxypropyl-beta-cyclodextrin, preferably as a cladribine-cyclodextrin complex, and excipients in the below given amounts:

| Name of ingredients | Formula mg/tablet |
|---|---|
| Cladribine-2- | 153.75 |
| hydroxypropyl-β- | equivalent to 10 mg 2-CdA |
| cyclodextrin- complex* | |
| Sorbitol powder | 44.25 |
| Magnesium Stearate | 2.0 |
| (vegetable grade) | |
| Total | 200.0 |

*Cladribine is complexed and lyophilised with 2-hydroxypropyl-beta-cyclodextrin in a separate process as described in WO 2004/087101, the disclosure of which is Incorporated by reference into this application in its entirety.

However, also similar compositions, preferably containing that cladribine-cyclodextrin complex, and preferably having the same or a very similar bioavailability, can preferably be used according to the invention.

If not explicitly defined otherwise, the naming of an active ingredient, active principle (API), medicament or international nonproprietary name (INN) thereof preferably includes all prodrugs, salts and solvates thereof, especially those that are functionally equivalent and/or are deemed a suitable substitute from a clinical point of view.

If not explicitly defined otherwise, the terms individuals, humans, human beings, human patients or patients are preferably used herein as interchangeable or as synonyms.

If not explicitly defined otherwise, the terms individual, human (being), human patient or (just) patient are preferably used herein as interchangeable or as synonyms.

Preferably in alignment with the wording in the Health Authority approved label/marketing authorisations, the term “treatment period” as used herein for the timeframe during which cladribine, preferably cladribine tablets, more preferably (Mavenclad®) is administered to subjects, preferably human subjects, more preferably patients and especially patients in need thereof, can be interchangeably also termed “treatment course” and/or “treatment cycle”. Accordingly, the terms “treatment period”, “treatment course” and/or “treatment cycle” can preferably used interchangeably within the context of the instant invention. The same holds preferably true for the terms “treatment periods”, “treatment courses” and/or “treatment cycles”. Preferably, if not explicitly defined otherwise, the terms “treatment period”, “treatment course” and/or “treatment cycle” can also be used, in accordance with the instant invention, in abbreviated form as “period”, “course” and/or “cycle”, respectively. However, in line with the USPI a, the term “First cycle” preferably means “month 1” in the “Year 1 treatment course” and/or “Year 2 treatment course”; likewise, the term “Second cycle” preferably means “month 2” in the “Year one treatment course” and/or “Year 2 treatment course”.

However, in the treatment methods described herein wherein the administration of the cladribine, preferably the administration of the oral cladribine and especially the administration of the cladribine tablets (e.g. Mavenclad) takes place according to or substantially in line with the posology as described in the EU SmPC/the proved European label, the two or more about a year long periods (also referred to as about 12 months, 46-54 weeks, 48-52 weeks, 48 weeks or 52 weeks, or the like) at the begin of which said cladribine, oral cladribine or cladribine tablets are administered, are preferably referred to as “treatment year” or “treatment years”, e.g. a “first treatment year”, a “second treatment year” or a “subsequent treatment year”. Likewise, in this context, each of the months, preferably each of the 2 months, at the beginning of a “treatment year”, in which months the cladribine, oral cladribine are cladribine tablets are administered to a patient are preferably referred to as “treatment course” or “treatment courses”, e.g. a “first treatment course”, a “second treatment course” or a “subsequent treatment course”.

In contrast thereto, in the treatment methods described herein wherein the administration of the cladribine, preferably the administration of the oral cladribine and especially the administration of the cladribine tablets (e.g. Mavenclad) takes place according to or substantially in line with the posology as described in the US PI/the approved US label, the two or more about a year long periods (also referred to as about 12 months, 46-54 weeks, 48-52 weeks, 48 weeks or 52 weeks, or the like) at the begin of which said cladribine, oral cladribine or cladribine tablets are administered, are preferably referred to as “treatment course” or “treatment courses”, e.g. a “first treatment course”, a “second treatment course” or a “subsequent treatment course”. Likewise, in this context, each of the about a month long periods, preferably each of the 2 about a month long periods, at the beginning of a “treatment course”, in which about a month long periods the cladribine, oral cladribine are cladribine tablets are administered to a patient are preferably referred to as “treatment cycles” or “treatment cycles”, e.g. a “first treatment cycle”, a “second treatment cycle” or a “subsequent treatment cycle”.

### REFERENCES

Black African and Latino/a identity correlates
with increased plasmablasts in MS
Kiel M. Telesford, PhD, Ulrike W. Kaunzner, MD, PhD, Jai Perumal, MD, Susan A. Gauthier, DO, MPH,
Xian Wu, MPH, Ivan Diaz, PhD, Mason Kruse-Hoyer, MD, Casey Engel, BA, Melanie Marcille, BA, and
Timothy Vartanian, MD, PhD
Neurol Neuroimmunol Neuroinflamm 2020; 7:e634. doi:10.1212NXI.0000000000000634
Immunization Responses in Rheumatoid Arthritis Patients
Treated With Rituximab
Clifton O. Bingham III,1 R. John Looney,2 Atul Deodhar,3 Neal Halsey,4
Maria Greenwald,5 Christine Codding,6 Benjamin Trzaskoma,7 Flavius Martin,?
Sunil Agarwal,7 and Ariella Kelman7
ARTHRITIS & RHEUMATISM
Vol. 62, No. 1, January 2010, pp 64-74
DOI 10.1002/art.25034
© 2010, American College of Rheumatology
Dysregulation of regulatory CD56bright NK cells/T cells interactions in
multiple sclerosis
Alice Laroni a, b, 1, Eric Armentani a, 1, Nicole Kerlero de Rosbo a, Federico Ivaldi a,
Emanuela Marcenaro b, c, Simona Sivori b, c, Roopali Gandhi d, Howard L. Weiner d, e, f,
Alessandro Moretta b, c, Giovanni L. Mancardi a, b, Antonio Uccelli a, b, *
Journal of Autoimmunity 72 (2016) 8-18
CD56bright Natural Killer Cells: A Possible Biomarker of Different Treatments in Multiple Sclerosis
Alice Laroni 1,2,* and Antonio Uccelli 1,2
J. Clin. Med. 2020, 9, 1450; doi:10.3390/jcm9051450 www.mdpi
Increased NK Cell Count in Multiple Sclerosis Patients Treated With Dimethyl Fumarate: A 2-Year Longitudinal Study
Damiano Marastoni 1†, Alessandro Buriani 2†, Anna Isabella Pisani 1, Francesco Crescenzol,
Carmela Zucol, Stefano Fortinguerra2, Vincenzo Sorrenti 2, Bruno Marenda2,
Chiara Romualdi 3, Roberta Magliozzi 1, Salvatore Monacol and Massimiliano Calabrese1*
Frontiers in Immunology|www.frontiersin.org|July 2019|Volume 10|Article 1666
Martin M D and Badovinac V P (2018)
Defining Memory CD8 T Cell.
Front. Immunol. 9:2692.
doi: 10.3389/fimmu.2018.02692
The Increased Ratio of Blood CD56bright NK to CD56dim NK
Is a Distinguishing Feature of Primary Sjögren's Syndrome
Bingxia Ming,1 Tong Wu,1 Shaozhe Cai,1 Peng Hu,1 Jungen Tang,1
Fang Zheng,2,3 Cong Ye,1 and Lingli Dong 1
1Department of Rheumatology and Immunology, Tongji Hospital, Tongji Medical College,
Huazhong
Hindawi
Journal of Immunology Research
Volume 2020, Article ID 7523914, 7 pages
https://doi.org/10.1155/2020/7523914
Using the Immunophenotype to Predict Response to
Biologic Drugs in Rheumatoid Arthritis
Ben Mulhearn 1,2,3,*, Anne Barton 2,3,4 and Sebastien Viatte 1,2,4
J. Pers. Med. 2019, 9, 46; doi:10.3390/jpm9040046 www.mdpi.com/journal/jpm
Regulatory B cells: the cutting edge of
immune tolerance in kidney transplantation
Bo Peng 1, Yingzi Mingl and Cheng Yang2
Peng et al. Cell Death and Disease (2018) 9:109
DOI 10.1038/s41419-017-0152-y
CD56bright natural killer (NK) cells: an important NK cell subset
Aure'lie Poli,1 Tatiana Michel,1 Maud The're'sine,1 Emmanuel Andre's,2 Francois Hentges1
and
Jacques Zimmer1
Immunology, 126, 458-465
Effect of rituximab treatment on T and B cell
subsets in lymph node biopsies of patients
with rheumatoid arthritis
Tamara H. Ramwadhdoebe1,2, Lisa G. M. van Baarsen1,2, Maria J. H. Boumans1,
Stefan T. G. Bruijnen 3, Mary Safy1, Ferco H. Berger4, Johanna F. Semmelink1,2,
Conny J. van der Laken3, Danielle M. Gerlag1,5, Rogier M. Thurlings1,a and
Paul P. Tak1,6,7,*
Rheumatology 2019; 58:1075-1085
doi:10.1093/rheumatology/key428
Advance Access publication 10 Jan. 2019
Rituximab treatment results in impaired secondary humoral immune
responsiveness
Lizet E. van der Kolk, Joke W. Baars, Martin H. Prins, and Marinus H. J. van Oers
BLOOD, 15 Sep. 2002_VOLUME 100, NUMBER 6
Natural killer cell subsets in cerebrospinal fluid of patients with
multiple sclerosis
E. Rodriguez-Martín,*‡§ C. Picón,*‡
L. Costa-Frossard,†‡§ R. Alenda,*‡§
S. Sainz de la Maza,† E. Roldán,*‡§
M. Espiño,*‡§ L. M. Villar*‡§ 1 and
J. C. Álvarez-Cermeliño†‡§ ¶1
Clinical and Experimental Immunology, 180: 243-249
B-cell populations discriminate between
pediatric- and adult-onset multiple sclerosis
Alexander Schwarz, MD; Bettina Balint, MD; Mirjam Korporal-Kuhnke, MD; Sven Jarius, MD; Kathrin von Engelhardt, MD; Alexandra Fürwentsches, MD; Cornelia Bussmann, MD; Friedrich Ebinger, MD; Brigitte Wildemann, MD*; Jürgen Haas, PhD*
Neurol Neuroimmunol Neuroinflamm 2017; 4:e309; doi: 10.1212/NXI.0000000000000309
Unique phenotypes and clonal expansions of
human CD4 effector memory T cells re-expressing CD45RA
Yuan Tian1, Mariana Babor1, Jerome Lane1, Veronique Schulten1, Veena S. Patil1, Grégory Seumois 1,
Sandy L. Rosales1, Zheng Fu 2, Gaelle Picarda3, Julie Burel1, Jose Zapardiel-Gonzalo1, Rashika N. Tennekoon1,4, Aruna D. De Silva1,4, Sunil Premawansa5, Gayani Premawansa6, Ananda Wijewickra
NATURE COMMUNICATIONS|8: 1473|DOI: 10.1038/s41467-017-01728-5|www.nature.com/naturecommunications
Regulatory B and T lymphocytes in multiple sclerosis: friends or foes?
Georgios K. Vasileiadis1 ⋅ Efthymios Dardiotis1 ⋅ Athanasios Mavropoulos2 ⋅ Zisis Tsouris1 ⋅ Vana Tsimourtou1 ⋅
Dimitrios P. Bogdanos2 ⋅ Lazaros I. Sakkas2 ⋅ Georgios M. Hadjigeorgiou1,3
Autoimmunity Highlights (2018) 9:9
https://doi.org/10.1007/s13317-018-0109-x
Cladribine—an old newcomer for pulsed immune reconstitution in MS
Heinz Wiendl
NATURE REVIEWS|NEUROLOGY VOLUME 13|OCTOBER 2017|573

All references cited herein are preferably hereby incorporated by reference in their entirety.

The invention is explained in greater detail below by means of examples. The invention preferably can be carried out throughout the range claimed and is not restricted to the examples given here.

Moreover, the following examples are given in order to assist the skilled artisan to better understand the present invention by way of exemplification. The examples are not intended to limit the scope of the invention.

### LEGEND OF THE FIGURES

FIG. 1: B cell differentiation pathways and expression of TrB-associated molecules.

FIG. 2: Main functions of human CD19+CD24hiCD38hi TrB cells discovered

FIG. 3. Study design of MAGNIFY-MS including the immune cell subtype analyses conducted as part of this sub-study. 9HPT, 9-hole peg test; BL, baseline; CUA, combined unique active; DMT, disease-modifying therapy; EDSS, Expanded Disability Status Scale; HIV, human immune deficiency virus; KFS, Kurtzke Functional System; MRI, magnetic resonance imaging; MS, multiple sclerosis; SDMT, symbol digit modalities test; T25FW, timed 25-foot walk; TB, tuberculosis.

FIG. 4. Median absolute values of B and T cell counts in patients treated with cladribine tablets. The first treatment course of cladribine tablets was administered at baseline and Month 1. BL, baseline; Q, quartile; TEMRA, terminally differentiated effector memory RA+; Th, T helper.

FIG. 5. Median absolute values of NK cell counts in patients treated with cladribine tablets. The first treatment course of cladribine tablets was administered at baseline and Month 1. BL, baseline; Q, quartile.

FIG. 6. Median serum concentration of immunoglobulins in patients treated with cladribine tablets. The first treatment course of cladribine tablets was administered at baseline and Month 1. BL, baseline; Ig, immunoglobulin; Q, quartile.

FIG. 7. Immune cells panels MAGNIFY-MS: Biomarkers for the identification of cell subsets

FIG. 8. Correlation matrix between numeric clinical attributes and B cell deconvolution scores. The areas of circles show the value of corresponding Pearson correlation coefficients. Positive correlations are displayed in blue and negative correlations in red. Color intensity (light to dark) and the size of the circle (small to big) are proportional to the correlation coefficients. Nonsignificant correlations (p-value<0.01) are marked with blank. Only EDDSS score at baseline is shown.

FIG. 9. Correlation matrix between numeric clinical attributes and CD8+ T cell deconvolution scores. The areas of circles show the value of corresponding Pearson correlation coefficients. Positive correlations are displayed in blue and negative correlations in red. Color intensity (light to dark) and the size of the circle (small to big) are proportional to the correlation coefficients. Nonsignificant correlations (pvalue<0.01) are marked with blank. Only EDDSS score at baseline is shown.

FIG. 10. Correlation matrix between numeric clinical attributes and CD4+ T cell deconvolution scores. The areas of circles show the value of corresponding Pearson correlation coefficients. Positive correlations are displayed in blue and negative correlations in red. Color intensity (light to dark) and the size of the circle (small to big) are proportional to the correlation coefficients. Nonsignificant correlations (p-value<0.01) are marked with blank. Only EDDSS score at baseline is shown.

FIG. 11. B cell subtypes—Absolute values displayed as cells/μL

FIG. 12. Immunoglobulins—Absolute values displayed as g/L

FIG. 13: T cell subtypes—Absolute values displayed as cells/μL

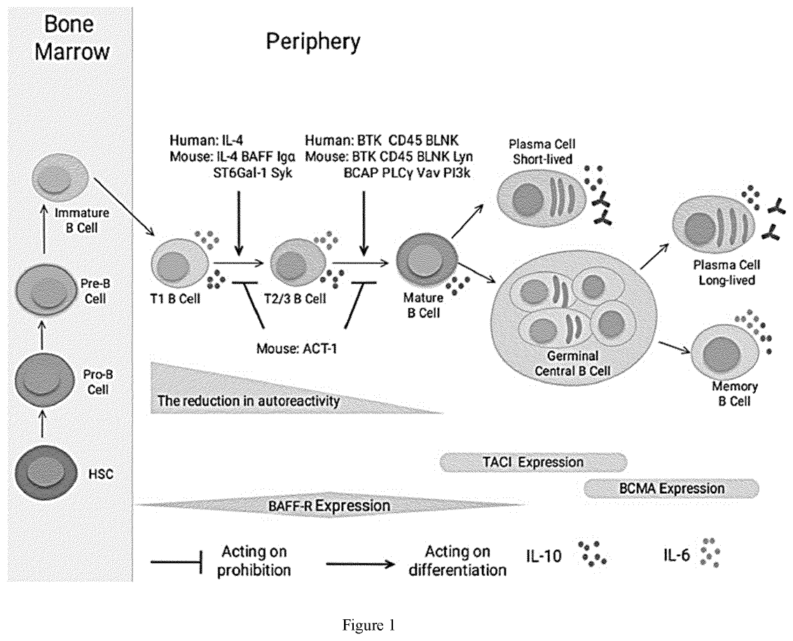

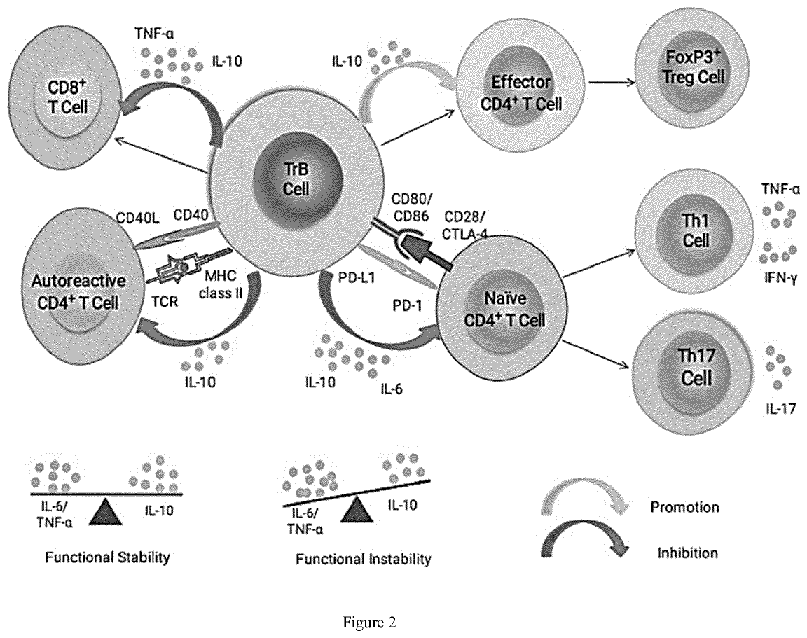

### EXAMPLES

### Example 1

Characterization of Peripheral Immune Cell Dynamics and Repopulation Patterns after Treatment with Cladribine Tablets in the MAGNIFY-MS Study

### Introduction

Multiple sclerosis (MS) is a chronic inflammatory disease of the central nervous system (CNS), in which many immune cell subtypes play a role in pathogenesis with B and T lymphocytes being involved in demyelination and axonal damage (Hoglund and Maghazachi, 2014; Piancone et al., 2016).
Cladribine tablets 10 mg (3.5 mg/kg cumulative dose over 2 years) is an immune reconstitution therapy (IRT) for MS that causes a selective and transient reduction in B and T cell counts, followed by a period of reconstitution (Giovannoni, 2017; Wiendl, 2017).
   Cladribine is a deoxyadenosine analog prodrug that is sequentially phosphorylated by deoxycytidine kinase (DCK) and deoxyguanosine kinase to its biologically active form, 2-chlorodeoxyadenosine triphosphate (Cd-ATP). The dephosphorylation and deactivation of Cd-ATP is catalyzed by 5′-NT-ase.
      B and T lymphocytes possess a relatively high DCK/5′-NT-ase ratio, making them particularly sensitive to cladribine (Giovannoni, 2017).
      Accumulation of cladribine within these immune cells leads to apoptosis through the inhibition of DNA polymerase. This effect on B and T cells interrupts the cascade of immune events that are central to the progression of MS (Deeks, 2018).
The long-term immune cell dynamics following treatment with cladribine tablets has been evaluated for CLARITY, CLARITY Extension, PREMIERE and ORACLE-MS studies (Comi et al., 2019; Stuve et al., 2019). But it remains to be fully established as to the effect on which immune cell subtypes may be key for both onset and durability of the therapeutic effect of cladribine tablets in people with MS.
   It is against this background that the MAGNIFY-MS (NCT03364036) was designed and initiated in those with highly active relapsing MS. Here we report findings for a substudy of peripheral immune cell subset dynamics and immunoglobulin levels during the first 12 months after patients had initiated treatment.

### Materials and Methods

### Study Design and Participants

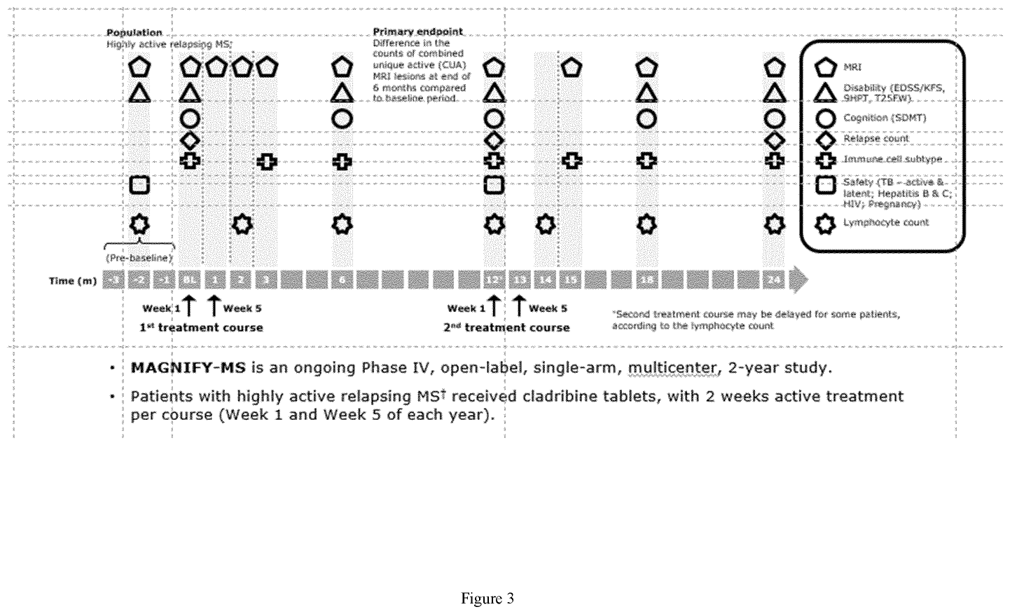

MAGNIFY-MS is an ongoing phase IV, open-label, single-arm study with a duration of 2 years (FIG. 3).
   Patients aged ≥18 years, with an Expanded Disability Status Scale (EDSS) score ≤5 and highly active relapsing MS were enrolled between May 28, 2018 and Apr. 23, 2019.
   In this study, highly active relapsing MS was defined as one relapse in the previous year and at least one T1 gadolinium enhancing (Gd+) lesion, or nine or more T2 lesions while on treatment with other disease-modifying therapies (DMTs), or two or more relapses in the previous year whether on DMT or not.
   Patients were excluded if they had previous exposure to DMT (fingolimod, natalizumab, alemtuzumab, mitoxantrone, or ocrelizumab), a lymphocyte count not within normal laboratory limits, presence of signs of progressive multifocal leukoencephalopathy, tested positive for human immunodeficiency virus, hepatitis B or C, or active/latent tuberculosis, had an active malignancy, or had an allergy or hypersensitivity to gadolinium and/or any other contraindication to perform magnetic resonance imaging (MRI).
Enrolled patients were scheduled to receive cladribine tablets 3.5 mg/kg cumulative dose over 2 years, with 2 weeks of active treatment per course (Week 1 and Week 5 of each year).

### Peripheral Blood Sampling

This exploratory analysis of MAGNIFY-MS involved longitudinal evaluation of peripheral blood immune cell subtypes in a subgroup of patients receiving cladribine tablets.
   Immunophenotyping was performed on blood samples collected at Baseline (pre-dose) and at the end of Months 3, 6, and 12.
      Further immunophenotyping was completed at Months 1 and 2 for TBNK and B cell panels.
      Immune cell subtypes were analysed by flow cytometry.
         Absolute cell counts and % change from baseline were assessed (Table 1).
In parallel, serum levels of immunoglobulins (Ig) G and M were analyzed by nephelometric assay.

### Statistical Analysis

The analysis of immune cell subtypes was an exploratory endpoint of the MAGNIFY-MS study, and were thus analysed descriptively. All patients that received at least one dose of cladribine tablets are included in the analysis.

### Results

Between May 28, 2018 and Apr. 23, 2019, a total of 70 patients were enrolled to this MAGNIFY-MS sub-study, and 57 of these patients received treatment with cladribine tablets (13 patients withdrew from the sub study prior to starting treatment).
The evaluable patient population was predominantly female (61.4%), aged ≤40 years (61.4%), and had a median EDSS score of 2.5 at baseline.
   Patient demographics and characteristics on entry to MAGNIFY-MS are shown in Table 2.

### B Cell Subtypes

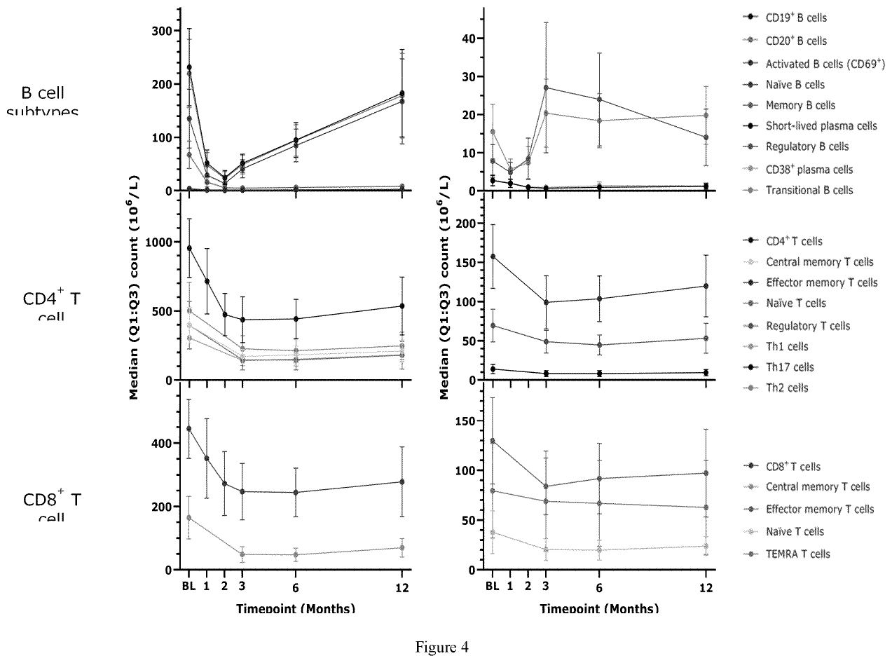

Changes in median CD19⁺, memory, and naïve B cell counts were relatively rapid, as shown by percentage change from baseline to Month 1 of −77%, −74%, and −80%, respectively (FIG. 4, Table 3).
Most B cell subtypes reached nadir by Month 2 after treatment, with CD38⁺ plasmablasts and short-lived plasma cells reaching nadir at Month 3.
   Thereafter, the reduction in memory B cells was sustained to Month 12.
   Regulatory B and transitional B cells recovered by Month 3, and increased over baseline levels.
   Naïve B cells also recovered to near baseline levels at 12 months after starting treatment.

### T Cell Subtypes

CD4⁺ and CD8⁺ T cells show a slow reduction in median cell counts, represented by a percentage change from baseline to Month 1 of −22% and −18%, respectively (FIG. 4, Table 3).
   Nadir was reached at Month 2, followed by a slight recovery toward baseline.
Other T cell subtypes reached nadir between Months 3 and 6, after which they slowly began to recover toward baseline.
Median Th17 and TEMRA T-cell counts show reductions at later time points, with Th17 reaching nadir at Month 6 (FIG. 4).

### Natural Killer Cells

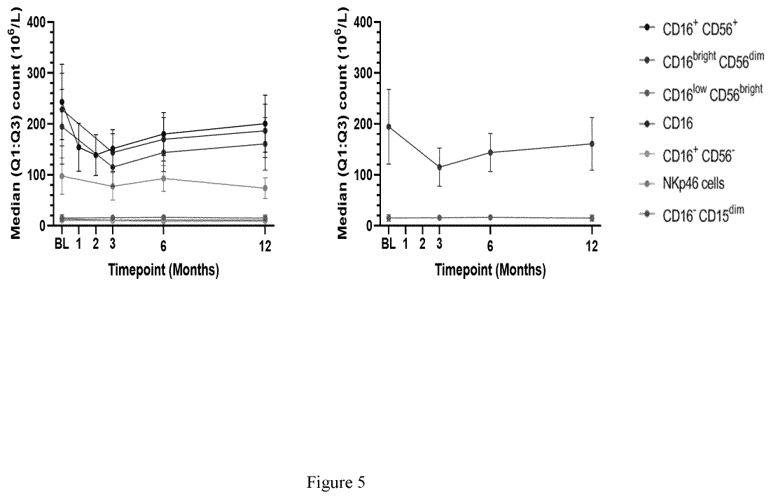

CD16⁺/CD56⁺ natural killer (NK) cell counts showed a decrease at Month 1 reaching nadir at Month 2 after treatment, represented by a change from baseline of −40% (Table 3, FIG. 5).
   Recovery of these subtypes towards baseline occurred from Months 3-12.
CD16^{bright} CD56^{dim} NK cell counts decreased to lowest levels at Month 3 after treatment, while no effect on CD16^{low} CD56^{bright} NK cell counts was noted.

### Immunoglobulins

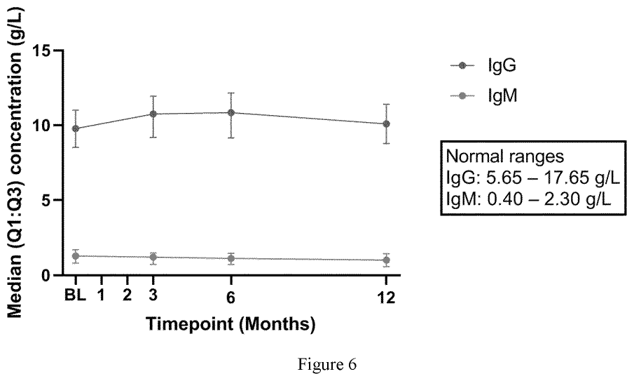

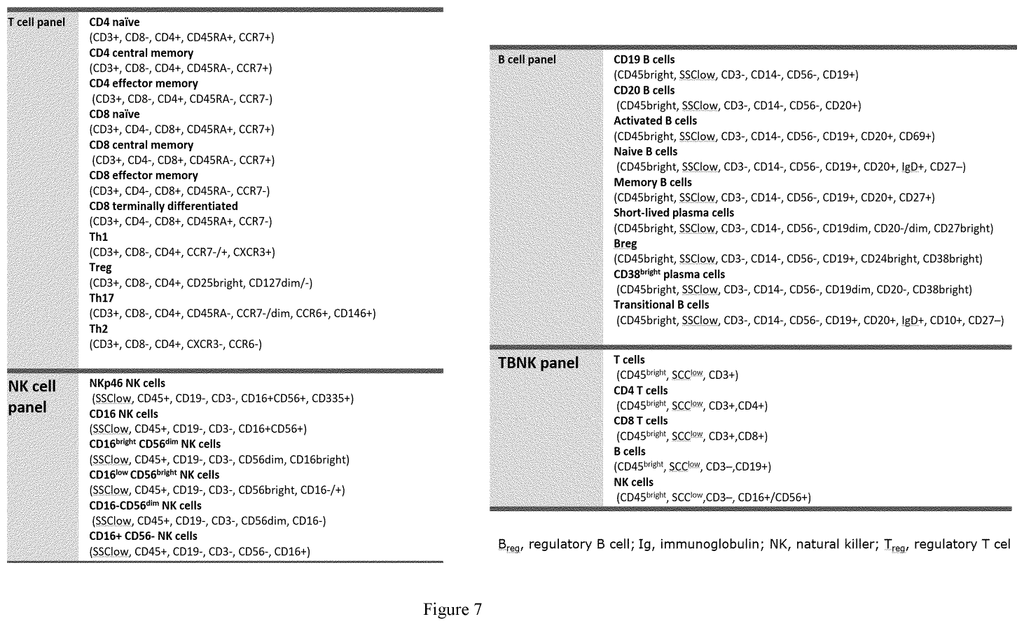

Despite the sustained decrease of memory B cells and a decrease in CD38⁺ plasma cells in the first year of MAGNIFY-MS, IgG and IgM levels remained within the normal range (FIG. 6).

### Intracellular Cytokines

### Discussion

The characterization of immune cell dynamics in response to disease-modifying treatment (DMT) may provide a better understanding of the pathogenesis of MS as well as providing key information on the mechanism of action of such therapy.
   This sub-study of the MAGNIFY-MS population assessed immune cell dynamics over the first year following treatment with cladribine tablets, thus enabling an evaluation of the onset of lymphocyte reduction and repopulation dynamics with specific focus on CD19⁺ B cells, CD4⁺ and CD8⁺ T cells, and CD16⁺ NK cell subtypes.
The dynamics of lymphocyte reduction indicate that B cells and NK cells reached nadir most rapidly, with almost all subtypes reaching nadir by Month 2.
   Most T cell subtypes reached nadir at Month 3, with CD8⁺ central memory, CD8⁺ effector memory, CD4⁺ and CD8⁺ naïve, and CD4⁺ regulatory cells reaching nadir at Month 6.
   For NK cells, the CD16^{low} CD56^{bright} cell subtype reached nadir at Month 12.
Regarding repopulation, most cell subtypes were recovering toward baseline by Month 12, with the exception of CD16^{low} CD56^{bright} NK cells.
   Perhaps surprising, given the reduction in memory B cells, is their sustained change from baseline at Month 12 in which there was an absolute percentage change of 6% from the nadir value of −93% at Month 2.
   However, whilst the median counts of memory B cells remain low, the numbers of naïve B cells had recovered toward baseline. This is important as it is these naïve B cells that are the main producers of interleukin-10, which may act to suppress undesired immune responses (Duddy et al., 2007).
The CD56^{bright} NK cell phenotype is regulatory (comprising 10% of the NK cell population) while CD56^{dim} (comprising 90% of the NK cell population) is effector. A decrease in the number of effector cells, in parallel with an increase in regulatory cells, will drive towards an anti-inflammatory environment and may therefore contribute to the therapeutic efficacy of cladribine tablets.
   Our results indicate that whilst CD56^{bright} cell counts were maintained there was a reduction in CD56^{dim} with nadir reached at Month 3. At this 3-month time point it could be assumed that an anti-inflammatory environment is achieved, although further analyses would need to be conducted to confirm this.
Our findings show that over the first year after treatment, the effects of cladribine tablets on B cells, T cells, and NK cells in patients with highly active relapsing MS are comparable to those observed in patients with clinically isolated syndrome and relapsing-remitting MS (Comi et al., 2019; Stuve et al., 2019).
We observed no clinically relevant changes in IgG and IgM concentration over the 12 month study period.
How do the results of this study compare to other DMTs?
   Alemtuzumab has been shown to decrease B cell, CD4⁺ and CD8⁺ T cell counts followed by a period of repopulation (Wiendl et al., 2020).
   The effects of alemtuzumab and cladribine was compared by Baker et al., and were found to be comparable with respect to B cell reduction (Baker et al., 2017).
   With fingolimod, total B cell counts were observed to decrease with treatment; an effect on naïve B cell counts was less apparent (Claes et al., 2014).
   B cell reduction following treatment with ocrelizumab is rapid, with reductions in CD19⁺ cell counts occurring as early as 2 weeks after treatment (Montalban et al., 2016).
      Note that direct comparisons of peripheral immune cell subset dynamics and Ig levels across studies should be made with caution due to differing assessment schedules and baseline patient characteristics.
Study strengths and limitations:
   Small sample size and a single-arm study, with no control group.
   Measurements for immune cell counts were made using only peripheral blood, yet cladribine is known to distribute into tissues such as those in the CNS shortly after administration (Hermann et al., 2019). This raises the possibility that this agent may act to reduce lymphocyte numbers within, and/or limit their recruitment into, the CNS as well as those circulating in the periphery, and further studies are warranted.
      Note how the immune cell subtype dynamics parallel the early onset of efficacy for MRI findings, reported separately.

### Conclusions

The early onset and thereafter sustained therapeutic effect of cladribine tablets may be mediated through a specific pattern of decrease and reconstitution of B and T cell subtypes.
   The pronounced effect on B cells, especially memory B cells in the first 2 months after initiation of cladribine tablets treatment, suggests a contribution to early efficacy onset.
   Sustained depletion of memory B cells and the moderate decrease across T cell subtypes may contribute to the long-term therapeutic effect of cladribine tablets.

TABLE 1
Cell subsets and phenotypes

| Lymphocyte subset | Phenotype |
|---|---|
| CD4 naïve T cells | (CD3⁺, CD8⁻, CD4⁺, CD45RA⁺, CCR7⁺) |
| CD4 central memory | (CD3⁺, CD8⁻, CD4⁺, CD45RA⁻, CCR7⁺) |
| T cells | |
| CD4 effector memory | (CD3⁺, CD8⁻, CD4⁺, CD45RA⁻, CCR7⁻) |
| T cells | |
| CD8 naïve T cells | (CD3⁺, CD4⁻, CD8⁺, CD45RA⁺, CCR7⁺) |
| CD8 central memory | (CD3⁺, CD4⁻, CD8⁺, CD45RA⁻, CCR7⁺) |
| T cells | |
| CD8 effector memory | (CD3⁺, CD4⁻, CD8⁺, CD45RA⁻, CCR7⁻) |
| T cells | |
| CD8 terminally | (CD3⁺, CD4⁻, CD8⁺, CD45RA⁺, CCR7⁻) |
| differentiated T cells | |
| Th1 cells | (CD3⁺, CD8⁻, CD4⁺, CCR7⁻/⁺, CXCR3⁺) |
| Treg cells | (CD3⁺, CD8⁻, CD4⁺, CD25^{bright}, CD127^{dim/-}) |
| Th17 cells | (CD3⁺, CD8⁻, CD4⁺, CD45RA⁻, |
| | CCR7⁻/^{dim}, CCR6⁺, CD146⁺) |
| Th2 cells | (CD3⁺, CD8⁻, CD4⁺, CXCR3⁻, CCR6⁻) |
| CD19 B cells | (CD45^{bright}, SSC^{low}, CD3⁻, CD14⁻, CD56⁻, |
| | CD19⁺) |
| CD20 B cells | (CD45^{bright}, SSC^{low}, CD3⁻, CD14⁻, CD56⁻, |
| | CD20⁺) |
| Activated B cells | (CD45^{bright}, SSC^{low}, CD3⁻, CD14⁻, |
| | CD56⁻, CD19⁺, CD20⁺, CD69⁺) |
| Naive B cells | (CD45^{bright}, SSC^{low}, CD3⁻, CD14⁻, |
| | CD56⁻, CD19⁺, CD20⁺, IgD⁺, CD27⁻) |
| Memory B cells | (CD45^{bright}, SSC^{low}, CD3⁻, CD14⁻, |
| | CD56⁻, CD19⁺, CD20⁺, CD27⁺) |
| Short-lived plasma | (CD45^{bright}, SSC^{low}, CD3⁻, CD14⁻, |
| cells | CD56⁻, CD19^{dim}, CD20⁻/^{dim}, CD27^{bright}) |
| Breg cells | (CD45^{bright}, SSC^{low}, CD3⁻, CD14⁻, |
| | CD56⁻, CD19⁺, CD24^{bright}, CD38^{bright}) |
| CD38^{bright} plasma cells | (CD45^{bright}, SSC^{low}, CD3⁻, CD14⁻, |
| | CD56⁻, CD19^{dim}, CD20⁻, CD38^{bright}) |
| Transitional B cells | (CD45^{bright}, SSC^{low}, CD3⁻, CD14⁻, CD56⁻, |
| | CD19⁺, CD20⁺, IgD⁺, CD10⁺, CD27⁻) |
| NKp46 NK cells | (SSC^{low}, CD45⁺, CD19⁻, CD3⁻, |
| | CD16⁺CD56⁺, CD335⁺) |
| CD16⁺ NK cells | (SSC^{low}, CD45⁺, CD19⁻, CD3⁻, |
| | CD16⁺CD56⁺) |
| CD16^{bright} CD56^{dim} | (SSC^{low}, CD45⁺, CD19⁻, CD3⁻, |
| NK cells | CD16^{bright}, CD56^{dim}) |
| CD16^{low} CD56^{bright} | (SSC^{low}, CD45⁺, CD19⁻, CD3⁻, CD16⁻/⁺, |
| NK cells | CD56^{bright}) |
| CD16⁻CD56^{dim} | (SSC^{low}, CD45⁺, CD19⁻, CD3⁻, CD16⁻ |
| NK cells | CD56^{dim}) |
| CD16⁺ CD56⁻ | (SSC^{low}, CD45⁺, CD19⁻, CD3⁻, |
| NK cells | CD56⁻, CD16⁺) |

Breg, B regulatory; NK natural killer; Treg, T regulatory.

TABLE 2
Patient demographics and characteristics
at the time of entry to MAGNIFY-MS.

| | Total Patients | |
|---|---|---|
| | (N = 57) | |
| Female, n (%) | 35 | (61.4) |
| Age in years, n (%) | | |
| ≤40 | 35 | (61.4) |
| >40 | 22 | (38.6) |
| Time since onset of MS in months, mean ± SD | 84.81 ± 93.54 | |
| Time since diagnosis in months, mean ± SD | 52.55 ± 67.45 | |
| Time since first relapse in months, mean ± SD | 52.62 ± 80.75 | |
| Number of relapses within 12 months prior to | | |
| baseline, n (%) | | |
| 0 | 2 | (3.5) |
| 1 | 15 | (26.3) |
| 2 | 29 | (50.9) |
| >2 | 11 | (19.3) |
| EDSS score at baseline, n (%) | | |
| ≤3 | 42 | (73.7) |
| >3 | 15 | (26.3) |
| Median (range) | 2.5 | (0.0-5.0) |

EDSS, Expanded Disability Status Scale; MS, multiple sclerosis; SD, standard deviation.

TABLE 3
Percentage change from baseline of B cell and T cell subtypes.

| | Month | Month | Month | Month | Month |
|---|---|---|---|---|---|
| Subtype | 1 | 2 | 3 | 6 | 12 |
| CD19⁺ B cells | −77% | −90% | −80% | −60% | −35% |
| | n = 46 | n = 44 | n = 46 | n = 35 | n = 42 |
| Memory B cells | −74% | −93% | −93% | −90% | −87% |
| | n = 45 | n = 44 | n = 46 | n = 34 | n = 42 |
| Activated B cells | −64% | −81% | −73% | −53% | −45% |
| (CD69⁺) | n = 45 | n = 44 | n = 46 | n = 34 | n = 42 |
| CD38⁺ plasma cells | −11% | −66% | −71% | −51% | −51% |
| | n = 45 | n = 44 | n = 46 | n = 34 | n = 42 |
| Short-lived plasma cells | −28% | −65% | −78% | −58% | −51% |
| | n = 45 | n = 44 | n = 46 | n = 34 | n = 42 |
| Naïve B cells | −80% | −90% | −75% | −43% | −5% |
| | n = 45 | n = 44 | n = 46 | n = 34 | n = 42 |
| Transitional B cells | −61% | −63% | +28% | +34% | +36% |
| | n = 45 | n = 44 | n = 46 | n = 34 | n = 42 |
| Regulatory B cells | −45% | −16% | +176% | +171% | +50% |
| | n = 45 | n = 44 | n = 46 | n = 34 | n = 42 |
| CD4⁺ T cells | −22% | −51% | −54% | −51% | −40% |
| | n = 46 | n = 44 | n = 46 | n = 35 | n = 42 |
| CD8⁺ T cells | −18% | −39% | −50% | −43% | −36% |
| | n = 46 | n = 44 | n = 46 | n = 35 | n = 42 |
| CD4⁺ Th1 cells | — | — | −51% | −45% | −35% |
| | | | n = 46 | n = 34 | n = 42 |
| CD4⁺ Th17 cells | — | — | −35% | −34% | −24% |
| | | | n = 45 | n = 34 | n = 41 |
| CD4⁺ CM/EffM T cells | — | — | −56%/−35% | −52%/−35% | −40%/−15% |
| | | | n = 46 | n = 34 | n = 42 |
| CD8⁺ CM/EffM T cells | — | — | −39%/−29% | −46%/−22% | −29%/−16% |
| | | | n = 46 | n = 34 | n = 42 |
| CD4⁺ naïve/CD8⁺ naïve | — | — | −63%/−73% | −64%/−70% | −53%/−58% |
| T cells | | | n = 46/n = 46 | n = 35/n = 34 | n = 42/n = 42 |
| CD4⁺ Treg cells | — | — | −30% | −38% | −21% |
| | | | n = 46 | n = 34 | n = 42 |
| CD8⁺ TEMRA | — | — | −26% | −15% | −19% |
| | | | n = 46 | n = 34 | n = 42 |
| CD16^{low} CD56^{bright} NK | — | — | −7% | −2% | −9% |
| cells | | | n = 46 | n = 34 | n = 42 |
| CD16⁺/CD56⁺ NK cells | −34% | −40% | −34% | −17% | −14% |
| | n = 46 | n = 44 | n = 46 | n = 35 | n = 42 |
| CD16^{bright} CD56^{dim} | — | — | −40% | −19% | −12% |
| NK cells | | | n = 46 | n = 34 | n = 42 |

Key:
Nadir value (in italics)
Values above baseline level (underlined)
CM, central memory; EffM, effector memory; NK, natural killer; TEMRA, terminally differentiated effector memory RA⁺; Th, T helper; Treg, T regulatory.

### Magnify-MS 1 Year Blood Biomarker Substudy: Methods

MAGNIFY-MS is an ongoing Phase IV (NCT03364036, see FIG. 3), open-label, single-arm, multicenter, 2-year study. Patients with highly active relapsing MS received cladribine tablets, with 2 weeks active treatment per course (Week 1 and Week 5 of each year). Subjects meeting the eligibility criteria during a pre-baseline screening period of up to −3 months before baseline (Day 1 of treatment) will receive an initial treatment course in Year 1 and a retreatment course in Year 2. Subjects will attend visits for assessments at end of Months 1, 2, 3, 6, 12 following Baseline (Day 1 of treatment) and at end of Month 15, 18, 24 following second treatment course (Month 1 of Year 2), respectively. Subjects will attend visits for blood sample as per mandatory monitoring at pre-Baseline and at Months 2, 6, 12, 14, 18 and 24. The primary endpoint(s) will be assessed during the first 6 months, secondary and tertiary endpoints will be assessed up to Month 24. Subjects will be offered the chance to participate in optional sub-studies for exploratory analysis.

To assess the effect of Mavenclad® on biomarkers in blood, an exploratory substudy has been planned to be completed in a subset patients. The goal of a blood biomarker substudy was to report on peripheral immune cell subset dynamics and immunoglobulin levels in the first 12 months of cladribine tablets therapy. The key objectives of performing exploratory research of markers of cladribine treatment response in MS are to define responders and non-responders to therapy and to better understand disease pathology. Validated biomarkers will help to define an optimal cladribine treatment response in MS and thus be of considerable value for taking treatment decisions and ensuring continued benefit from cladribine therapy. A reliable marker of cladribine treatment response in MS should correlate with cladribine bioactivity, be implicated in MS pathology and thus correlate with disease activity or severity. Given inter-subject variability and heterogeneity of MS pathology the combined analysis of several different markers will be most meaningful.

This blood biomarker sub-study of MAGNIFY-MS involved longitudinal evaluation of peripheral blood immune cells in patients receiving cladribine tablets. 57 patients were treated from the 70 patients enrolled. Whole blood and Peripheral mononuclear cells (PBMCs) were collected at months 1, 2, 3, 6, and 12 and Immunophenotyping was performed using the FACS (Fluorescence-activated cell sorting) technology. Absolute cell counts and % change from baseline were assessed for each immune cell subtype. Additional primary and secondary trial readouts are illustrated in FIG. 3. In addition to the described changes in biomarker over time the correlation of biomarkers with ‘traditional’ MS related assessments (e.g. Relapse, Mill, EDSS) will be performed. The action of cladribine tablets on immune cells may be key for both onset and durability of its effect in people with MS.

### Blood Analysis

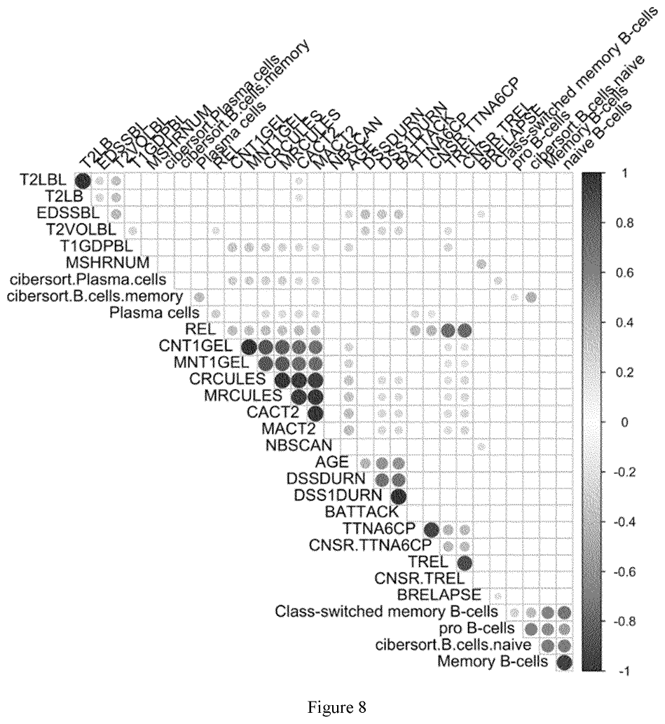

Blood will be analysed for immune cell subtypes and intracellular cytokines using the FACS technology. Blood samples will be drawn specifically for the evaluation of potential immunological surrogate biomarker (see also FIG. 8). In order to distinguish different immune subtypes the following FACS Marker Panels were validated and applied by Covance.

### T Cell Panel:

CD4 Naïve, CD4 Central Memory, CD4 Effector Memory, CD8 Naïve, CD8 Central Memory, CD8 Effector Memory, CD8 Terminally Differentiated, Th1, Treg, Th17, Th2

### B Cell Panel

CD19 B cells, CD20 B cells, Activated B cells, Naïve B cells, Memory B cells, Short-lived Plasma cells, Breg, CD38^{bright} Plasma cells, Transitional B cells

### NK Cells Panel

NKp46 NK cells, CD16 NK cells, CD16bright CD56dim NK cells, CD16low CD56bright NK cells, CD16-CD56dim NK cells, CD16+CD56− NK cells

### TBNK Panel

T cells (CD45bright, SCClow, CD3+), CD4 T cells (CD45bright, SCClow, CD3+,CD4+), CD8 T cells (CD45bright, SCClow, CD3+,CD8+), B cells (CD45bright, SCClow, CD3−,CD19+), NK cells (CD45bright, SCClow, CD3−, CD16+/CD56+)
   The following intracellular cytokines may be analysed/measured.
T cells cytokines: IL-10, IL-17, GM-CSF, TNF-a, IFN-γ, IL-22, IL-4
B cells cytokines: IL-10, IL-6, GM-CSF

### Example 2

### Correlation of Plasma Cells and T Cells with MRI Efficacy at the Clarity 2-Year Timepoint (as Newly Determined from CLARITY 96 Weeks Study)

Plasmacell Correlation with MRI

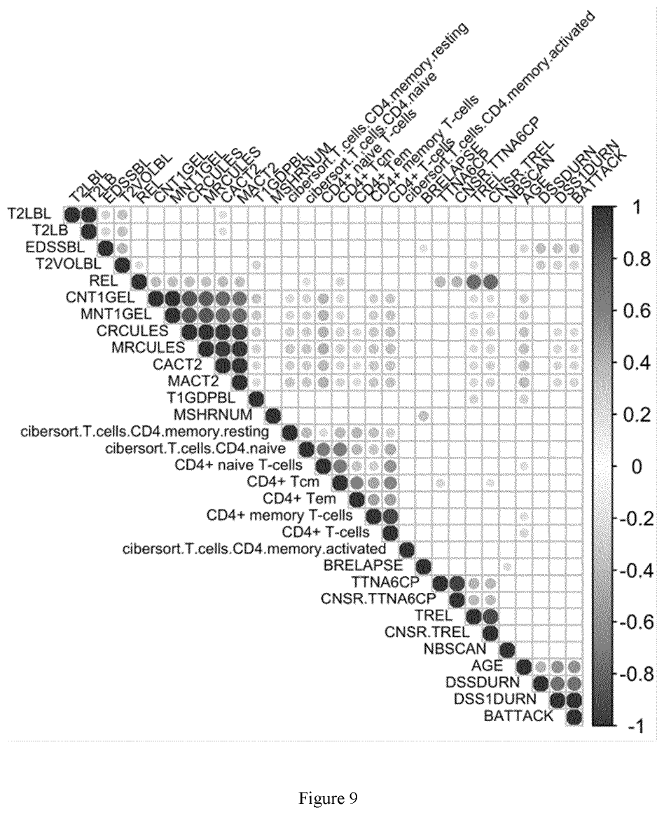

Plasma cell signatures have weak positive, but significant, correlation with: MACT2 (Mean Active T2 Lesions), CACT2 (Cumulative Active T2 lesions), MRCULES (Mean CU Lesions), MNT1GEL (Mean New T1 Gd+ Lesions). The results are illustrated in FIG. 9.

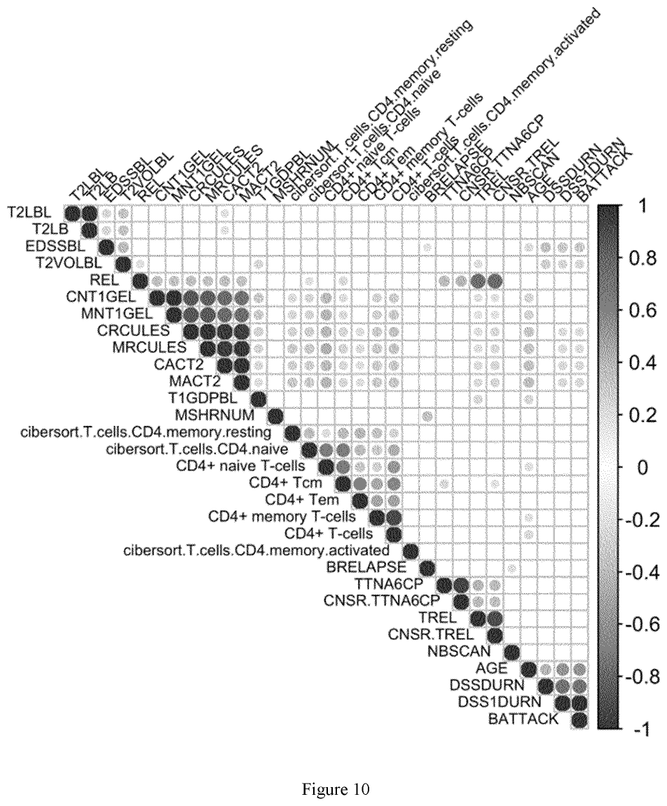

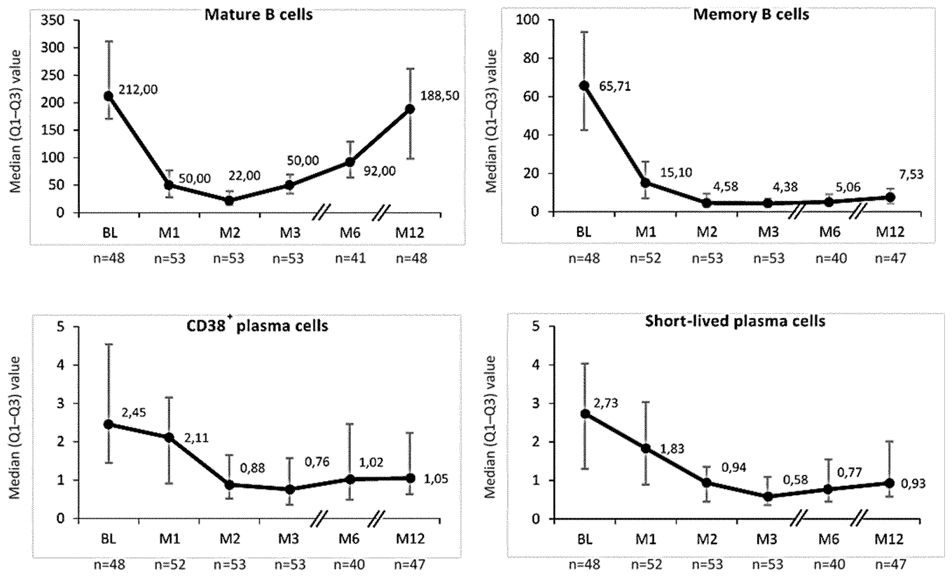

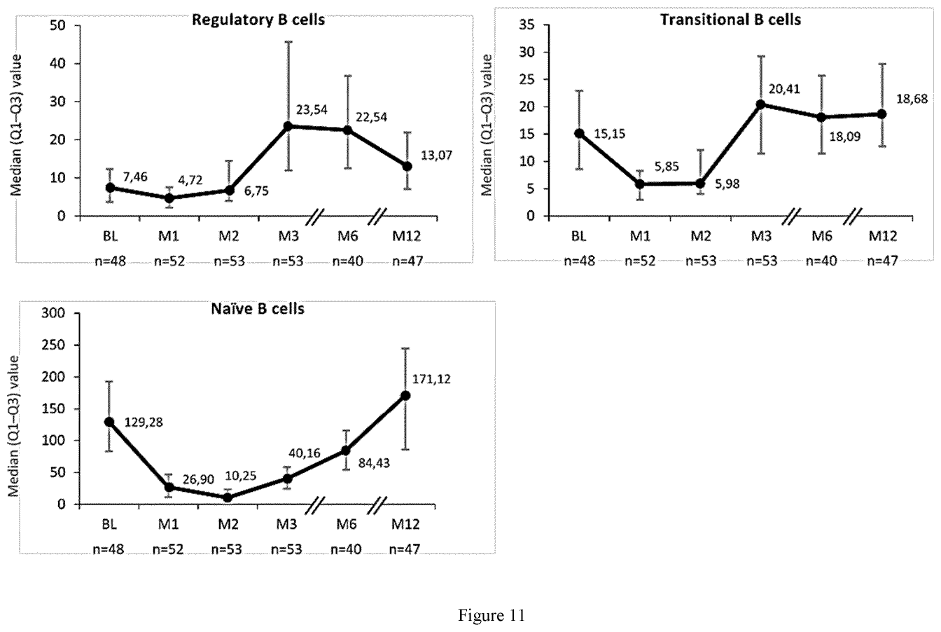

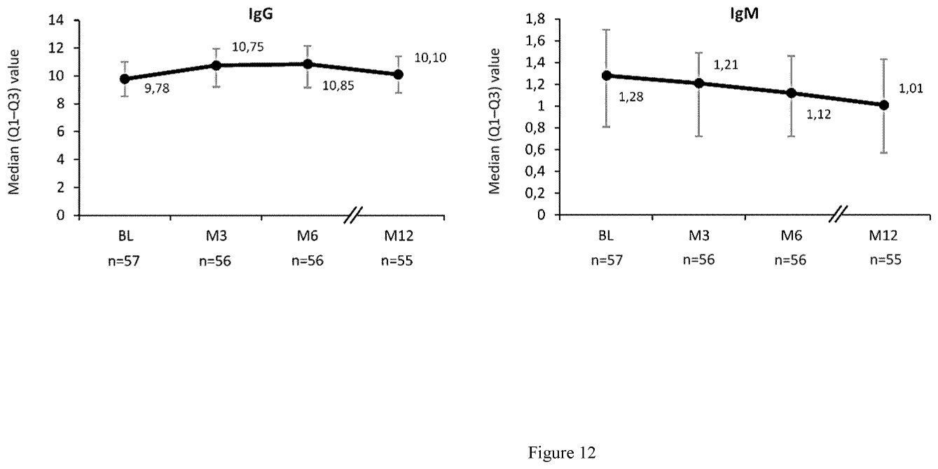

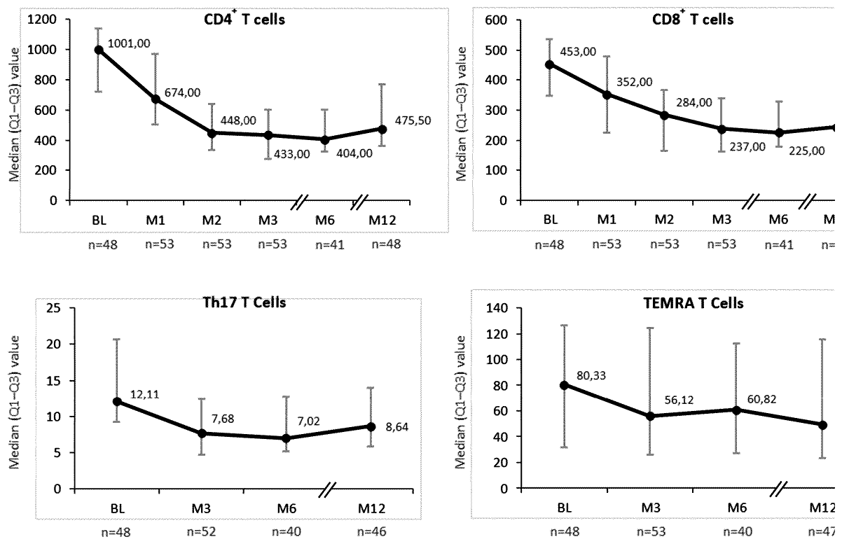

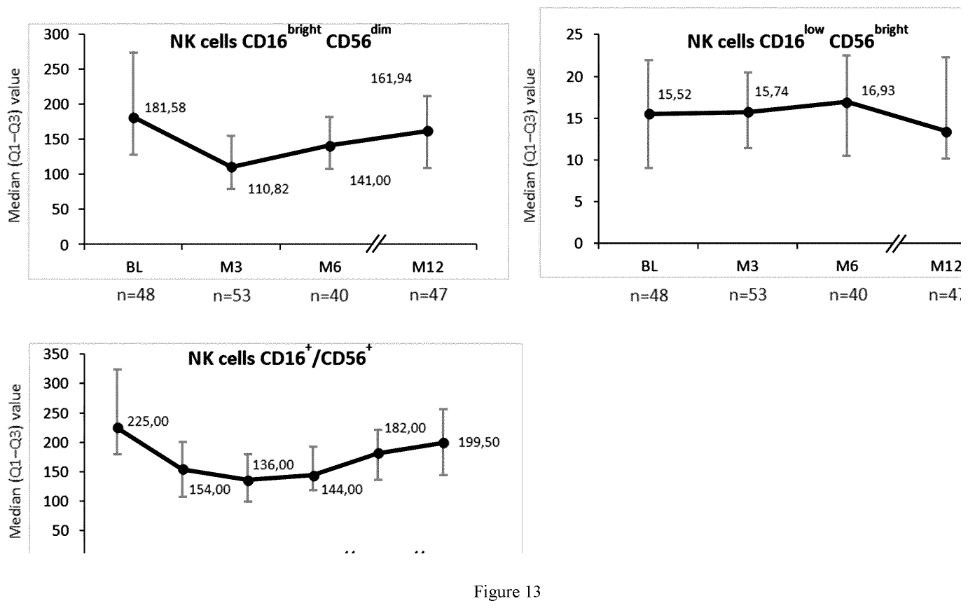

CD8⁺ T Cell Correlation with MRI
Naive CD8⁺T cells, CD8⁺ T-cell and CD8⁺ Tem signatures have positive correlation with:
   CNT1GEL (Cumulative New T1 Gd+ Lesions), MNT1GEL (Mean New T1 Gd+ Lesions), CRCULES (Cumulative CU Lesions), MRCULES (Mean CU Lesions), CACT2 (Cumulative Active T2 Lesions), MACT2 (Mean Active T2 Lesions). The results are illustrated in FIG. 10.
   Naïve CD4⁺ and CD4⁺CM T Cell Signatures have Positive Correlation with MRI:
most of the CD4+ cell related signature correlate well with MRI attributes—especially CD4⁺ naive T cells
REL (number of relapses) is associated with naive CD4⁺ T-cell signature from CIBERSORT and CD4+ Tcm (from xCell). The results are illustrated in FIG. 11.

TABLE 1a
Percentage change from baseline of B cell subtypes

| | Month | Month | Month | Month | Month |
|---|---|---|---|---|---|
| Subtype | 1 | 2 | 3 | 6 | 12 |
| CD19⁺ B cells | −77% | −90% | −80% | −60% | −35% |
| | n = 46 | n = 44 | n = 46 | n = 35 | n = 42 |
| Memory B cells | −74% | −93% | −93% | −90% | −87% |
| | n = 45 | n = 44 | n = 46 | n = 34 | n = 42 |
| CD38⁺ plasma cells | −11% | −66% | −71% | −51% | −51% |
| | n = 45 | n = 44 | n = 46 | n = 34 | n = 42 |
| Short-lived plasma | −28% | −65% | −78% | −58% | −51% |
| cells | n = 45 | n = 44 | n = 46 | n = 34 | n = 42 |
| Naïve B cells | −80% | −91% | −75% | −43% | −5% |
| | n = 45 | n = 44 | n = 46 | n = 34 | n = 42 |
| Transitional B cells | −61% | −63% | +28% | +34% | +36% |
| | n = 45 | N = 44 | n = 46 | n = 34 | n = 42 |
| Regulatory B cells | −45% | −16% | +176% | +171% | +50% |
| | n = 45 | n = 44 | n = 46 | n = 34 | n = 42 |

Key:
NA, Not Available
Nadir value in bold
Values above baseline level

TABLE 1b
Percentage change from baseline of T cell subtypes

| | Month | Month | Month | Month | Month |
|---|---|---|---|---|---|
| Subtype | 1 | 2 | 3 | 6 | 12 |
| CD4⁺ | −22% | −51% | −54% | −51% | −50% |
| | n = 46 | n = 44 | n = 46 | n = 35 | n = 42 |
| CD8⁺ | −18% | −39% | −50% | −43% | −36% |
| | n = 46 | n = 44 | n = 46 | n = 35 | n = 42 |
| CD4⁺ Th1 | NA | NA | −51% | −45% | −35% |
| | | | n = 46 | n = 34 | n = 42 |
| CD4⁺ Th17 | NA | NA | −35% | −34% | −24% |
| | | | n = 45 | n = 34 | n = 41 |
| CD4⁺ CM/EffM | NA | NA | −56%/−35% | −53%/−35% | −40%/−15% |
| | | | n = 46 | n = 34 | n = 42 |
| CD8⁺ CM/EffM | NA | NA | −39%/−29% | −46%/−22% | −29%/−16% |
| | | | n = 46 | n = 34 | n = 42 |
| CD4⁺ naïve/CD8⁺ naïve | NA | NA | −63%/−73% | −69%/−70% | −53%/−58% |
| | | | n = 46/n = 46 | n = 35/n = 34 | n = 42/n = 42 |
| CD4⁺ Treg | NA | NA | −30% | −38% | −21% |
| | | | n = 46 | n = 34 | n = 42 |
| CD8⁺ TEMRA | NA | NA | −26% | −15% | −19% |
| | | | n = 46 | n = 34 | n = 42 |

TABLE 1c
Percentage change from baseline of NK cell subtypes

| | Month | Month | Month | Month | Month |
|---|---|---|---|---|---|
| Subtype | 1 | 2 | 3 | 6 | 12 |
| CD16^{low} CD56^{bright} | NA | NA | −7% | −2% | −9% |
| natural killer cells | | | n = 46 | n = 34 | n = 42 |
| CD16⁺/CD56⁺ | −34% | −40% | −34% | −17% | −14% |
| natural killer cells | n = 46 | n = 44 | n = 46 | n = 35 | n = 42 |
| CD16^{bright} CD56^{dim} | NA | NA | −40% | −19% | −12% |
| natural killer cells | | | n = 46 | n = 34 | n = 42 |

### Table Legend:

The percentage change from baseline was measured for B (1a), T (1b) and NK cell subtypes.
There is early onset of action, with most B cell subtypes reaching nadir levels by Month 2. CD19+, Memory and naïve B cells show an early onset at Month 1. Short lived plasma cells and C38⁺ plasma cells decrease at Month 2 and reach nadir at Month 3. In contrast transitional and regulary B cells are reduced up 61% and 45% at month 1 and then increase reaching levels higher than the basal values measured.
T cell subtypes show a in general a similar profile in reduction with nadir values for CD4⁺ And CD8⁺ subtypes at Month 3 and 6 respectively. Interestingly regulatory CD4+ (Treg) and CD8+(TEMRA) shows a less prominent decrease of 38% and 26%.
NK cell subtypes

1.-7. (canceled)

8. A method to predict the response of a patient treated with cladribine for the treatment of an autoimmune disorder,
a) wherein deviations of cell populations of:
CD19⁺B cells of at least 70%,
memory CD19⁺B cells of at least 70%,
naïve B cells of at least 70%,
memory B cells of at least 70%,
activated B cells, and/or
activated CD69+ B cells of at least 45%,
and/or combinations thereof;
are indicative of early efficacy of said cladribine treatment;
wherein:
cell populations are determined in one or more body fluids samples of said patient at different stages of said cladribine treatment including determining said cell population in a body fluids sample from the patient collected before the start of treatment with cladribine and determining said cell population in a body fluids sample from the patient collected at at least a later point in time during or after treatment with cladribine,
said deviations are given as decrease from the baseline levels;
b) wherein deviations of cell populations of:
CD19⁺B cells of at least 70%,
CD38+ Plasma cells of at least 50%,
Short lived plasma ells of at least 50%,
Bregs of at least 70%,
transitional B cells of at least 70%,
naïve T cells of at least 10%,
T central memory cells of at least 40%,
T central effector cells of at least 30%,
TH1 cells of at least 35%,
TH17 cells of at least 35%,
and/or combinations thereof;
are indicative of durable efficacy of said cladribine treatment at 1 year;
wherein:
cell populations are determined in one or more body fluids samples of said patient at different stages of said cladribine treatment including determining said cell population in a body fluids sample from the patient collected before the start of treatment with cladribine and determining said cell population in a body fluids sample from the patient collected at at least a later point in time during or after treatment with cladribine,
said deviations are given as decrease from the baseline levels;
c) wherein:
a deviation of cell populations of memory B cells to a level above −40%,
a deviation of cell populations of plasma cells to a level above −20%,
a deviation of cell populations of Tnaive cells to a level above −40%,
a deviation of cell populations of CD4+ Central memory T cells to a level above −20%,
a deviation of cell populations of CD4+ Effector Memory T cells to a level above −10%
a deviation of cell populations of the Treg/Teff cell ratio wherein the deviation is a decrease,
a deviation of cell populations of the Breg/B memory cell ratio wherein the deviation is a decrease,
a deviation of cell populations of the NKreg/NK eff cell ratio wherein the deviation is a decrease,
and/or combinations thereof,
are indicative of a risk of reactivation of multiple sclerosis;
wherein:
cell populations are determined in one or more body fluids samples of said patient at different stages of said cladribine treatment including determining said cell population in a body fluids sample from the patient collected before the start of treatment with cladribine and determining said cell population in a body fluids sample from the patient collected at at least a later point in time during or after treatment with cladribine,
said deviations are from the baseline levels.

9. The method of claim 8, wherein the autoimmune disorder is multiple sclerosis.

10. A method for monitoring the need for adapting the treatment of an autoimmune disorder with a Disease Modifying Drug (DMD) of a patient in need thereof,
wherein the patient has been treated with a DMD which is cladribine for the treatment of an autoimmune disorder,
wherein deviations of cell populations as determined in one or more body fluids samples of said patient at different stages of said cladribine treatment including determining said cell population in a body fluids sample from the patient collected before the start of treatment with cladribine and determining said cell population in a body fluids sample from the patient collected at at least a later point in time during or after treatment with cladribine are selected from the list consisting in:
a deviation of cell populations of memory B cells to a level above −40%,
a deviation of cell populations of plasma cells to a level above −20%,
a deviation of cell populations of Tnaive cells to a level above −40%,
a deviation of cell populations of CD4+ Central memory T cells to a level above −20%,
a deviation of cell populations of CD4+ Effector Memory T cells to a level above −10%,
a deviation of cell populations of the Treg/Teff cell ratio wherein the deviation is a decrease,
a deviation of cell populations of the Breg/B memory cell ratio wherein the deviation is a decrease,
a deviation of cell populations of the NKreg/NK eff cell ratio wherein the deviation is a decrease,
and/or combinations thereof;
wherein said deviations are indicative of a need for a retreatment of said patient with cladribine.

11. The method of claim 10, wherein the autoimmune disorder is multiple sclerosis.

12. A method for the treatment of an autoimmune disorder with a Disease Modifying Drug (DMD) of a patient at risk of reactivation of multiple sclerosis,
wherein the patient has been treated with a DMD which is cladribine for the treatment of an autoimmune disorder,
wherein deviations of cell populations as determined in one or more body fluids samples of said patient at different stages of said cladribine treatment including determining said cell population in a body fluids sample from the patient collected before the start of treatment with cladribine and determining said cell population in a body fluids sample from the patient collected at at least a later point in time during or after treatment with cladribine are selected from the list consisting in:
a deviation of cell populations of memory B cells to a level above −40%,
a deviation of cell populations of plasma cells to a level above −20%,
a deviation of cell populations of Tnaive cells to a level above −40%,
a deviation of cell populations of CD4+ Central memory T cells to a level above −20%,
a deviation of cell populations of CD4+ Effector Memory T cells to a level above −10%,
a deviation of cell populations of the Treg/Teff cell ratio wherein the deviation is a decrease,
a deviation of cell populations of the Breg/B memory cell ratio wherein the deviation is a decrease,
a deviation of cell populations of the NKreg/INX eff cell ratio wherein the deviation is a decrease,
and/or combinations thereof;
are indicative that the patient is a patient at risk of reactivation of multiple sclerosis,
wherein said treatment comprises administering to the patient a retreatment with cladribine, the administration of an additional and/or higher dose of cladribine or the administration of a different DMD.

13. The method of claim 12, wherein the autoimmune disorder is multiple sclerosis.

14. The method of claim 12, wherein the retreatment with cladribine comprises the oral administration of a formulation comprising cladribine, wherein the formulation is to be orally administered following the sequential steps below:
(i) an induction period wherein said cladribine formulation is administered and wherein the total dose of cladribine reached at the end of the induction period is from about 1.7 mg/kg to about 3.5 mg/kg;
(ii) a cladribine-free period of between about 8 and about 10 months wherein no cladribine formulation is administered;
(iii) a maintenance period wherein said cladribine formulation is administered and wherein the total dose of cladribine reached at the end of the maintenance period is lower than the total dose of cladribine reached at the end of the induction period (i); and
(iv) a cladribine-free period wherein no cladribine formulation is administered.

15. A method of monitoring the response of a patient having an autoimmune disease chosen among autoimmune neurological and neuroinflammatory diseases, to treatment with a disease-modifying drug (DMD) said method comprising:
a) obtaining the absolute numbers of any of:
total PBMCs;
CD19⁺ B cells;
CD19⁺ memory B cells;
naive B cells;
CD4⁺ Central memory T cells;
CD4⁺ Effector memory T cells;
Treg;
Teff;
Breg;
memory B cells;
CD16^{dim} CD56^{bright} NK cells;
CD16^{bright} CD56^{dim} NK cells;
transitional B cells;
in a first and a second biological sample from the patient,
wherein any of:
an increase of the absolute number of CD19+ B cells or a decrease of 30% or less of the absolute number of CD19+ B cells, in the second biological sample compared to the first biological sample;
an increase of the absolute number of CD19+ memory B cells or a decrease of 30% or less of the absolute number of CD19+ memory B cells, in the second biological sample compared to the first biological sample; and/or
an increase of the absolute number of naive B cells or a decrease of 30% or less of the absolute number of naive B cells, in the second biological sample compared to the first biological sample;
an increase of the absolute number of CD4+ Central memory T cells or a decrease of 20% or less of the absolute number of CD4+ Central memory T cells, in the second biological sample compared to the first biological sample;
an increase of the absolute number of CD4⁺ Effector memory T cells or a decrease of 10% or less of the absolute number of CD4⁺ Effector memory T cells, in the second biological sample compared to the first biological sample;
a decrease in the ratio of the absolute number of Treg over the absolute number of Teff in the second biological sample compared to said ratio in the first biological sample;
a decrease in the ratio of the absolute number of Breg cells over the absolute number of memory B cells in the second biological sample compared to said ratio in the first biological sample;
—a decrease in the ratio of the absolute number of CD16^{dim} CD56^{bright} NK cells over the absolute number of CD16^{bright} CD56^{dim} NK cells in the second biological sample compared to said ratio in the first biological sample;
a percentage of memory B cells over total PBMCs of between 0.05% and 1% in the second biological sample;
a percentage of Breg cells over total PBMCs of between 3% and 14% in the second biological sample;
a ratio of Transitional B cells:memory B cells of superior or equal to 1, in the second biological sample;
is indicative of a sub-optimal response of the patient to the treatment.

16. The method of claim 15, wherein the autoimmune disease is multiple sclerosis.

17. A method of treating a patient having an autoimmune disease chosen among autoimmune neurological and neuroinflammatory diseases, to treatment with a disease-modifying drug (DMD) said method comprising:
a) obtaining a first biological sample from the patient,
b) administering the treatment to the patient;
c) obtaining a second biological sample from the patient;
d) determining in the first and the second biological sample the absolute numbers of any of:
total PBMCs;
CD19⁺ B cells;
CD19⁺ memory B cells;
naive B cells;
CD4⁺ Central memory T cells;
CD4⁺ Effector memory T cells;
Treg;
Teff;
Breg;
memory B cells;
CD16^{dim} CD56^{bright} NK cells;
CD16^{bright} CD56^{dim} NK cells;
transitional B cells;
wherein any of:
an increase of the absolute number of CD19+ B cells or a decrease of 30% or less of the absolute number of CD19+ B cells, in the second biological sample compared to the first biological sample;
an increase of the absolute number of CD19+ memory B cells or a decrease of 30% or less of the absolute number of CD19+ memory B cells, in the second biological sample compared to the first biological sample; and/or
an increase of the absolute number of naive B cells or a decrease of 30% or less of the absolute number of naive B cells, in the second biological sample compared to the first biological sample;
an increase of the absolute number of CD4+ Central memory T cells or a decrease of 20% or less of the absolute number of CD4+ Central memory T cells, in the second biological sample compared to the first biological sample;
an increase of the absolute number of CD4⁺ Effector memory T cells or a decrease of 10% or less of the absolute number of CD4⁺ Effector memory T cells, in the second biological sample compared to the first biological sample;
a decrease in the ratio of the absolute number of Treg over the absolute number of Teff in the second biological sample compared to said ratio in the first biological sample;
a decrease in the ratio of the absolute number of Breg cells over the absolute number of memory B cells in the second biological sample compared to said ratio in the first biological sample;
a decrease in the ratio of the absolute number of CD16^{dim} CD56^{bright} NK cells over the absolute number of CD16^{bright} CD56^{dim} NK cells in the second biological sample compared to said ratio in the first biological sample;
a percentage of memory B cells over total PBMCs of between 0.05% and 1% in the second biological sample;
a percentage of Breg cells over total PBMCs of between 3% and 14%;
a ratio of Transitional B cells:memory B cells of superior or equal to 1, in the second biological sample;
is indicative of a sub-optimal response of the patient to the treatment,
administering to the patient with a sub-optimal response to the treatment a retreatment with cladribine, an additional and/or higher dose of cladribine or the administration of a different DMD.

18. The method of claim 17, wherein the retreatment with cladribine comprises the oral administration of a formulation comprising cladribine, wherein the formulation is to be orally administered following the sequential steps below:
(i) an induction period wherein said cladribine formulation is administered and wherein the total dose of cladribine reached at the end of the induction period is from about 1.7 mg/kg to about 3.5 mg/kg;
(ii) a cladribine-free period of between about 8 and about 10 months wherein no cladribine formulation is administered;
(iii) a maintenance period wherein said cladribine formulation is administered and wherein the total dose of cladribine reached at the end of the maintenance period is lower than the total dose of cladribine reached at the end of the induction period (i); and
(iv) a cladribine-free period wherein no cladribine formulation is administered.

19. The method of claim 17, wherein the autoimmune disease is multiple sclerosis.

20. A method of treating an autoimmune disorder in a patient in need thereof, said method comprising orally administering cladribine to said patient in an amount of 0.5 to 1.25 mg per kilogram of body weight of said patient in a time period between within 4 to 12 weeks, wherein, after said administration, said patient is having one or more deviations with regard to one or more of the cell populations, selected from the group consisting of:
a) wherein the following deviations are biomarkers/correspond to early efficacy of said cladribine treatment, given as % decrease from the baseline levels:
CD19⁺B cells (>70%+/−10%),
memory CD19⁺B cells (>70%+/−10%),
naïve B cells (>70%+/−10%),
memory B cells (>70%+/−10%),
activated B cells, and/or
activated CD69⁺ B cells (>45+/−10%),
and/or combinations thereof;
b) Durable efficacy at 1 year (% decrease from baseline):
memory CD19+,
B cells (>70%+/−10%),
CD38+ Plasmacells (>50%+/−10%),
Short lived plasmacells (>50%+/−10%),
Bregs (>70%+/−10%),
transitional B cells (>70%+/−10%),
naïve T cells (>10%+/−5%),
T central memory cells (>40%+/−10%),
T central effector cells (>30%+/−10%),
TH1 cells (>35+/−10%),
TH17 cells (>35+/−10%),
and/or combinations thereof;
c) Durable efficacy:
a NKreg/NKeff cell ratio (>1.5 fold),
a Breg/Beff cell ratio (>/<fold),
a Treg/Teff cell ratio (>/<fold), and/or
d) Reactivation of disease
decrease of Memory B cells from baseline (<30%+/−10%),
memory B cells (0.05 and 1% of PBMCs),
a transitional B cell ratio in total PBMC,
Bregs in whole blood or PBMCs from active or reactivating MS patients (3-14%), and/or
e) Reactivation of Disease:
memory B cells (repopulating to a level above −40% from baseline),
plasma cells (repopulating to a level above −20% from baseline),
Tnaive cells (repopulating to a level above −40% from baseline),
CD4+ Central memory T cells (repopulating to a level above −20% from baseline),
CD4+ Effector Memory T cells (repopulating to a level above −10% from baseline),
a Treg/Teff cell ratio (decreased from baseline), and/or
a Breg/B memory cell ratio (decreased compared to baseline), and/or
a NKreg/NK eff cell ratio (decreased compared to baseline);
and/or combinations thereof.

21. The method of claim 20, wherein the autoimmune disorder is multiple sclerosis.
